(19) United States
(12) Patent Application Publication
(10) Pub. No.: US 2026/0242439 A1
(43) Pub. Date: Aug. 20, 2026

(54) RECOMBINANT HUMAN PENTRAXIN-1/PENTRAXIN-2 (RHPTX-1/2) FUSION PROTEINS FOR THE TREATMENT OF INFECTIOUS DISEASES AND OTHER PATHOLOGIES

(71) Applicant: W. Scott WILLETT, Doylestown, PA (US)

(72) Inventor: W. Scott WILLETT, Doylestown, PA (US)

(21) Appl. No.: 19/539,891

(22) Filed: Feb. 13, 2026

Related U.S. Application Data
(60) Provisional application No. 63/758,353, filed on Feb. 14, 2025.

(51) Int. Cl. C07K 14/705; A61K 39/12; A61K 39/145; A61K 47/68; A61P 37/04; C07K 16/00; G01N 33/58

(52) U.S. Cl. CPC C07K 14/705; A61K 39/12; A61K 39/145; A61K 47/6801; A61P 37/04; C07K 16/00; G01N 33/581; G01N 33/582; C07K 2319/00

(57) ABSTRACT

Fusion proteins of either rhPTX-1 or rhPTX-2 (rhPTX-1/2) designed to diagnose, treat, or prevent viral or other pathogenic infectious or cancerous diseases, or to mitigate blood-borne toxicities; methods for preparing rhPTX-1/2 fusion proteins; pharmaceutical compositions including rhPTX-1/2 fusion proteins, and methods of use are described herein.

### PRIORITY CLAIM AND CROSS-REFERENCED TO RELATED APPLICATION

This application claims the benefit of U.S. Provisional Application No. 63/758,353, filed Feb. 14, 2025, which application is expressly incorporated by reference herein in its entirety.

### FIELD OF THE INVENTION

Provided herein is a novel fusion protein platform comprising pentraxins. The platform enables the formation of unique fusion proteins based on pentraxins such as Pentraxin 1 or Pentraxin 2, enabling the creation of new biopharmaceuticals uniquely and custom designed to diagnose, treat, or prevent viral or other pathogenic infectious, cancerous, or immune system diseases, or to mitigate blood-borne toxicities.

### SEQUENCE LISTING

The instant application contains a Sequence Listing which has been submitted electronically in XML format and is hereby incorporated by reference in its entirety. Said XML copy, created on Feb. 12, 2026, is named J3596-00002_SL.xml, and is 21,922 bytes in size.

### BACKGROUND OF THE INVENTION

The large-scale production of therapeutic proteins was enabled by the advent of recombinant DNA technology in the 1970s. The first recombinant protein drug was human insulin, introduced in 1982. Since then, the biopharmaceutical industry has developed thousands of recombinant protein drugs, with more than 200 currently available as commercial products (Usmani S S, Bedi G, Samuel J S, Singh S, Kalra S, Kumar P, et al. (2017) PLOS ONE 12 (7): e0181748). Today FDA-approved biopharmaceuticals include recombinant hormones, interferons, interleukins, hematopoietic growth factors, tumor necrosis factors, blood-clotting factors, thrombolytic drugs, enzymes, monoclonal antibodies, and vaccines. The majority of protein therapeutics on the market are antibodies, constructed with the IgG class of immunoglobulins known as monoclonal antibodies (mAbs). While these mAb products have been shown to be safe and effective, they are complex multimeric (two light chains, two heavy chains) molecules that can be difficult to develop, and costly to manufacture. Additionally, IgGs possess complex immune effector functions via Fcγ receptors, and complement system interactions via C1q binding (Liu, R. et al, 2020, Antibodies 2020, 9, 64; doi: 10.3390/antib9040064) that are in part dependent on the glycosylation structures attached to N297 in the Fc region of the molecules. Because glycosylation patterns of recombinantly-produced mAbs are a major source of product structural and functional heterogeneity, much effort has been spent trying to understand and engineer the Fc domains in order to control off-target effects of this class of therapeutics.

In 1989 a serendipitous discovery of single-chain antibodies from camelids (camels, llamas, alpacas) revealed an alternative to monoclonal antibodies (Arbadi-Ghahroudi, et al, 2017, Frontiers in Immunology 8 Article 1589). Camelid single-chain antibodies, designated V_{H}H domains and also known as nanobodies, are small (130-140 aa, ~15 kDa), monomeric, non-glycosylated binding domains that have been shown to have high binding affinities similar to IgGs, yet with greater versatility in binding applications. As a result of this discovery, there are many groups developing V_{H}H-based therapeutics and diagnostics.

Camelid single-chain antibodies offer several advantages over monoclonal antibodies. They are only about 15 kDa, and are not glycosylated, making them readily manufacturable in many industrial expression systems, including both microbial and mammalian cells. They are thermodynamically stable, enabling long-term storage, are readily soluble, and strictly monomeric. The antigen binding regions of V_{H}Hs are simpler, and structurally distinct from those of IgGs, resulting in high affinity binding to both large and small molecules and smaller inaccessible epitopes, unlike IgGs that bind preferentially to larger structures and poorly to small molecules and structurally constricted epitopes. However, due to their small size, native V_{H}H domains naturally have very short half-lives in human plasma following IV injection, limiting their effectiveness as therapeutics. Therefore, making effective therapeutics from V_{H}Hs will require either making multimers or creating protein-carrier conjugates of these binding domains to increase their apparent size above ~50 kDa (glomerular filtration limit). Another advantage of camelid V_{H}Hs relative to IgGs is the ease of generating libraries of binders from which to select drug candidates. Due to the monomeric, single-domain character of V_{H}Hs, direct cloning of V_{H}H repertoires into phage display platforms can be used for screening and selection, in contrast to generating Fab or scFv libraries that involve artificially linking heavy and light chain variable regions where the natural VH-VL pairings resulting from immunization can be lost (Arbadi-Ghahroudi, et al, 2017, ibid). Additionally, universal VHH framework region scaffolds have been developed, enabling randomization and grafting of the 3 complementarity determining regions (CDRs), thus facilitating VHH library construction and screening of product candidates (Liu, B., 2022, Int. J. Mol. Sci 23 1482).

Recombinant human Pentraxin-2 (rhPTX-2) has previously been evaluated for the treatment of fibrosis (zinpentraxin alpha, NCT04594707). Pentraxin-2 is a human plasma protein that functions as part of the innate immune system and is a member of the Pentraxin family of proteins, together with Pentraxin-1 or C-reactive protein (PTX-1, CRP). Pentraxins are known as a pattern recognition receptors that, via bound calcium ions, non-specifically bind to and facilitate clearance of danger-related and pathogen-related molecular patterns (DAMPS and PAMPS) from the body. Although the inherent biological activities of PTX-1 and PTX-2 are apparently not identical, for the purposes of this invention, the short Pentraxins 1 and 2 will be considered interchangeable in the fusion protein designs described below and will be referred to as rhPTX-1/2. Upon calcium-mediated opsonization of DAMPS or PAMPS on one face of the pentamer, Pentraxin-2 interacts with cells of the immune system on the opposite face of the pentamer by binding to Fcγ or Fcα receptors to ameliorate inflammatory signaling, helping to resolve inflammatory/fibrotic pathogenesis, leading to tissue homeostasis. Conversely, PTX-1 (CRP) is an acute-phase protein associated with inflammatory responses following injury. The FcR binding epitopes on rhPTX-1/2-opsonized materials bind to and crosslink Fcγ or Fcα receptors on immune cells such as dendritic cells, macrophages, and monocytes to facilitate phagocytosis and stimulation of adaptive (T-cells and B-cells) immune responses (Lu, J, et al, 2008, Nature 456:989-992; Barahwaj, D, et al, 2001, J. Immunol. 166:6735-6741; Castaño, A. P., 2009, Science Trans. Med., 1:5, 5ra13, Breedveld, A., 2019, Frontiers in Immunol 10:553; Lu, J., 2014, Protein Science 23:378-386). Fcγ receptors, specifically FcγRI, FcγRIIa, and FcγRIII, have been shown to be critical mediators of adaptive immune responses following viral and bacterial infections (Herrada, A. A., et al, 2007 PNAS 104:33, pp 13402-13407; Huber, V. C., et al, 2001, J. Immunol., 166:7381-7388; Saeland, E., et al, 2003, J. Infectious Diseases, 187:1686-93; International Patent #WO 2019/125846 A1, Bournazos, S. and Ravetch, J). Thus, engineering the binding of rhPTX-1/2 variants to selected FcγR or FcαR, either towards higher or lower affinity of a specific receptor subtype, thus tuning the immune signaling of the engineered PTX fusion, will likely result in improved therapeutics and vaccines designed to treat infectious diseases (Bournazos, S. et al, 2020 Nature; Ko, S. et al, 2021, BioDrugs). Pentraxin-2 is a homopentamer (FIG. 1) and a member of the pentraxin family of proteins. All pentraxins share a common core structure as non-covalent pentamers, comprising 5 identical protomers arranged in a disc shape. The pentraxin family is further divided into two groups: short, and long pentraxins. Pentraxin-1 (CRP) and pentraxin-2 (aka serum amyloid P component, or SAP) are short pentraxins, with each protomer possessing approximately 225 amino acids (protomer MW=~25 kDa, pentamer MW=~125 kDa). Pentraxin-3 is one example of a long pentraxin, which differs from short pentraxins by having an N-terminal extension of 160 amino acids. The natural existence of both long and short pentraxins suggests creating active, stable N-terminal fusions to short pentraxins such as hPTX-1 or hPTX-2 is feasible. In fact, this is already described for rhPTX-2 in patent application US 2014/0302024 where an anti-TNFα V_{H}H3 domain was fused to the N-terminus (creating a Pentabody). This novel fusion protein was shown to possess both anti-TNF and hPTX-2 biological activity in the context of creating an enhanced antifibrotic drug. Importantly, the potency of the pentameric anti-TNFα Pentabody was shown to be approximately 1000-fold more potent in a cell-based bioassay than its commercial IgG comparator, REMICADE®, which also binds TNFα (see application US 2014/0302024, FIG. 11)

By way of structural comparison, there is another naturally occurring pentameric form of antibody: Immunoglobulin M (IgM). Along with IgGs, IgM is part of the adaptive immune system. IgMs are considered the oldest class of immunoglobulin and are produced transiently during early stages of an infection before IgG responses predominate. IgMs are very large molecules (>500 kDa) produced at low abundance in the body (5-10% of all immunoglobulins vs. 80% IgG) that provide short-term low-affinity multivalent antigen recognition. Notably however, IgM's are very complex molecules that are difficult and costly to produce at large scale in an industrial setting, whereas routine production of commercially relevant amounts of rhPTX-1/2 has been established. Fusion proteins of rhPTX-1/2 present the potential for an eloquent bridge between the innate and adaptive immune systems that may result in very potent, highly effective vaccines, therapeutics, and diagnostics.

What is needed are novel therapeutics that enable a bridge between the innate and adaptive immune systems resulting in very potent, highly effective vaccines, therapeutics, and diagnostics. What is particularly needed are novel therapeutics comprising biopharmaceuticals that couple the inherent innate immune system functionality of pentraxins, including but not limited to, rhPTX-1/2 with targeted pentavalent broad spectrum molecular binding, or with pentavalent antigen or other selected bioactivity presentation, wherein such biopharmaceuticals are designed for therapeutic use, to prevent or treat infectious disease or other blood-borne pathogens and toxins, as potentially enable cancer therapies.

### SUMMARY OF THE INVENTION

Embodiments of this invention are generally directed to the therapeutic use of recombinant human pentraxin-1/2 (rhPTX-1/2) as a platform technology for the multivalent presentation of targeted binding domains, antigens, or other selected bioactivities related to a fusion partner. The platform structure of this invention comprises a fusion protein comprising a fusion partner at the N- or C-terminus of PTX-1/2, followed by or preceded by rhPTX-1/2. In certain embodiments, the fusion protein comprises a fusion partner at the N-terminus, followed by a peptide linker for domain separation, rhPTX-1/2 at the C-terminus. In certain other embodiments, the fusion protein comprises a fusion partner at the C-terminus, preceded by a peptide linker for domain separation, and rhPTX-1/2 at the N-terminus. These fusion protein designs are also known as an N- or C-terminal fusion to rhPTX-1/2. This platform configuration allows for any protein sequence to be linked to the rhPTX-1/2 protomer in a way that preserves the structure and function of each fusion protein domain. Because rhPTX-1/2 is a naturally formed stable pentamer, this invention enables the production of engineered pentameric antibodies, antigens, or other selected bioactivities, including covalently coupled or non-covalently complexed small molecules with desired pharmacological properties. Fusion partners may include, but are not limited to, enzymes, hormones, growth factors, neuropeptides, internalization peptides, cell-signaling factors, cytokines, toxins, inhibitors, and activators, light-emitting proteins, agonists, antagonists, or annexins.

As used herein, human Pentraxin-1 (hPTX-1) is equivalent to human C-reactive protein or hCRP.

As used herein, rhPTX-1 comprises recombinant human Pentraxin-1.

As used herein, human Pentraxin-2 (hPTX-2) is equivalent to Serum Amyloid P Component, or hSAP.

As used herein, rhPTX-2 comprises recombinant human Pentraxin-2.

As used herein, the term rhPTX-1/2 refers to recombinant human Pentraxin 1 or recombinant human Pentraxin 2.

As used herein, the term “protomer” refers to the one of the amino acid chains that constitutes the pentameric pentraxin-related fusion protein. Accordingly, a protomer includes rhPTX-1/2 linked to a fusion partner (rhPTX-1/2 fusion protein). Pentraxin molecules assembled from protomers can be either homopentameric or heteropentameric if either a single protomer or a combination of different protomers are expressed simultaneously in the cell.

In certain aspects, the invention is directed to a composition comprising an rhPTX-1 or rhPTX-2 pentamer that has from 1-5 protomers that are identical N-terminal or C-terminal fusion proteins, or an rhPTX-1/2 (1/2 denotes either C Reactive Protein (CRP) or SAP pentraxins) pentamer comprising from 1 to 5 distinct fusion proteins. For the sake of brevity, only N-terminal fusion designs are depicted herein, although C-terminal fusion designs are possible and may have the advantage of facilitating multimeric presentation of fusion partners whose native conformations are dimers, trimers, quatromers, or pentamers.

In certain aspects, the invention is directed to a composition of rhPTX-1/2 fusion protein comprising from 1-5 unmodified rhPTX-1/2 protomers.

While hPTX-1 is not naturally glycosylated, in certain aspects, the invention is directed to a fusion protein composition comprising 1-5 modified rhPTX-2 protomers that do not contain a glycosylation consensus sequence at position 32 such that the rhPTX-2 moiety of the fusion protein is not glycosylated.

In certain aspects, the invention is directed to a composition of rhPTX-1/2 fusion protein wherein the rhPTX-1/2 moiety of the fusion protein is at least 80%, 85%, 90%, 95%, 96%, 97%, 98%, 99%, or 100% identical in sequence to native rhPTX-1 or rhPTX-2 as shown in SEQ ID NO. 1 (CRP) and SEQ ID NO. 2 (SAP).

In certain aspects, the invention is directed to an rhPTX-1/2 fusion protein that contains one or more peptide(s) or chemical linker(s) between the fusion partner and the rhPTX-1/2 moiety (SEQ ID NOs. 1 or 2).

In certain aspects, the invention is directed to an rhPTX-1/2 fusion protein that contains a modified rhPTX-1/2 sequence that enables chemical attachment of a linker and/or fusion partner.

In certain aspects, the invention is directed to a composition of rhPTX-1/2 comprising fusion to a binding domain wherein the binding domain is targeted to a viral protein or viral antigen.

In certain aspects, the invention is directed to a composition of rhPTX-1/2 comprising fusion to an antibody binding domain wherein the binding domain is targeted to a bacterial or microbial protein or microbial antigen.

In certain aspects, the invention is directed to a composition of rhPTX-1/2 comprising fusion to a binding domain wherein the binding domain is targeted to a small molecule toxin or hapten.

In certain aspects, the invention is directed to a composition of rhPTX-1/2 comprising fusion to an antigen or antigen fragment wherein the antigen is selected from a virus, microbe, or other infectious agent. These rhPTX-1/2 fusion proteins are known as PENTAGENS™ and are used as vaccines to elicit specific immune responses to the presented antigen.

In certain aspects, the invention is directed to a composition of rhPTX-1/2 comprising fusion to a protein with selected bioactivity, including but not limited to, enzymes (PENTAZYME™), hormones, growth factors, neuropeptides, cell-signaling factors, cytokines (PENTAKINE™), inhibitors, antagonists, or agonists.

In certain aspects, the invention is directed to a composition of rhPTX-1/2 comprising fusion to camelid V_{H}H domain, lectin (carbohydrate binding proteins), or annexin (Ca⁺²-dependent lipid binding proteins). These rhPTX-1/2 fusion proteins are referred to as PENTABODIES™.

In certain aspects, the invention is directed to pharmaceutical preparations of rhPTX-1/2 fusion proteins as described herein, with one or more pharmaceutically-acceptable formulations or excipients for delivery by intravenous (IV) injection, intramuscular (IM) injection, intraperitoneal (IP) injection, subcutaneous injection, intravitreal injection, inhalation through the nose or mouth via a nebulizer or dry-powder inhaler, or topical application to the dermis, eye, or lung.

In certain aspects, the invention provides methods for preparing rhPTX-1/2 fusion proteins in either a microbial or mammalian host cell comprising 1) generating a production cell line using current molecular biological methods (recombinant DNA technology) where all five protomers are identical, or 2) generating a cell line capable of expression of from 1-5 distinct protomers by introduction of 1-5 distinct rhPTX-1/2 fusion protein gene sequences into the production cell line, including native rhPTX-1/2 protomers with no fusion partner.

In certain aspects, the invention is directed to methods for treating patients with effective doses of a selected rhPTX-1/2 fusion protein. In some embodiments, the rhPTX-1/2 fusion protein (PENTABODY™) is administered to treat an active viral or microbial infection in order to reduce and eliminate the infectious agent from the patient's blood, or to mitigate the pathogenic effects of the infectious or toxic agent. In some embodiments, the rhPTX-1/2 fusion protein (PENTABODY™) is administered to reduce or to clear a toxin or other molecular pathogen from the patient's blood. In some embodiments, the rhPTX-1/2 fusion protein (PENTABODY™) is administered to crosslink selected cell types to induce or facilitate cell signaling. In some embodiments, the rhPTX-1/2 fusion protein (PENTAGEN™) is administered as a vaccine against a targeted viral, microbial, or other infectious agent. In some embodiments, the rhPTX-1/2 fusion protein (PENTAZYME™) is administered to deliver a selected enzymatic activity to a patient. In some embodiments, the rhPTX-1/2 fusion protein is administered to a patient to deliver a selected biological activity such as a cytokine (PENTAKINE™), or as described herein.

In certain embodiments, it may be advantageous to bind and crosslink more than one antigen or pathogen causing a disease state in a patient in order to maximize the potency of an rhPTX-1/2 fusion protein. In certain aspects, the invention is directed to a method of treating or preventing infectious disease with a heteropentameric rhPTX-1/2 fusion protein consisting of distinct protomers containing from 1-5 different binding domains targeting different antigens resulting in intramolecular multivalency, or INTRAVALENCY™.

In some situations, it may be advantageous to present more than one antigen on one molecule in order to maximize the potency of an rhPTX-1/2 fusion protein vaccine (PENTAGEN™). In certain aspects, the invention is directed to methods for vaccinating against infectious disease with a heteropentameric rhPTX-1/2 fusion protein comprising from 1-5 distinct protomers presenting different antigens resulting in intramolecular multivalency, or INTRAVALENCY™.

### Protein Sequences

SEQ ID NO. 1: Native hPTX-1 (C-reactive protein; CRP), Gene bank Accession
No. M11725.1
QTDMEKLLCFLVL TSLSHAFGQT DMSRKAFVFP KESDTSYVSL KAPLTKPLKA
FTVCLHFYTE LSSTRGYSIF SYATKRQDNE ILIFWSKDIG YSFTVGGSEI LFEVPEVTVA
PVHICTSWES ASGIVEFWVD GKPRVRKSLK KGYTVGAEAS IILGQEQDSF
GGNFEGSQSL VGDIGNVNMW DFVLSPDEIN TIYLGGPFSP NVLNWRALKY
EVQGEVFTKP QLWP
SEQ ID NO. 2: Native hPTX-2 (Serum Amyloid P Component; SAP), Gene bank
Accession No. NP_001630
HTDLSGKVFV FPRESVTDHV NLITPLEKPL QNFTLCFRAY SDLSRAYSLF
SYNTQGRDNE LLVYKERVGE YSLYIGRHKV TSKVIEKFPA PVHICVSWES
SSGIAEFWIN GTPLVKKGLR QGYFVEAQPK IVLGQEQDSY GGKFDRSQSF
VGEIGDLYMW DSVLPPENIL SAYQGTPLPA NILDWQALNY EIRGYVIIKP LVWV
SEQ ID NO. 3: Fusion protein platform design: Fusion Partner-Linker-
rhPTX-2
Fusion Partner-Linker-HTDLSGKVFV FPRESVTDHV NLITPLEKPL QNFTLCFRAY
SDLSRAYSLF SYNTQGRDNE LLVYKERVGE YSLYIGRHKV TSKVIEKFPA
PVHICVSWES SSGIAEFWIN GTPLVKKGLR QGYFVEAQPK IVLGQEQDSY
GGKFDRSQSF VGEIGDLYMW DSVLPPENIL SAYQGTPLPA NILDWQALNY
EIRGYVIIKP LVWV
SEQ ID NO. 4: Fusion protein no linker design: Fusion Partner-rhPTX-2
Fusion Partner-HTDLSGKVFV FPRESVTDHV NLITPLEKPL QNFTLCFRAY
SDLSRAYSLF SYNTQGRDNE LLVYKERVGE YSLYIGRHKV TSKVIEKFPA
PVHICVSWES SSGIAEFWIN GTPLVKKGLR QGYFVEAQPK IVLGQEQDSY
GGKFDRSQSF VGEIGDLYMW DSVLPPENIL SAYQGTPLPA NILDWQALNY
EIRGYVIIKP LVWV

The human Pentraxin-1 sequence in SEQ ID NO. 1 is the full-length, native human sequence. The human Pentraxin-2 sequence in SEQ ID NO. 2 is the full-length, native human sequence. For the purpose of brevity, the fusion proteins in this present invention can be constructed from either rhPTX-1 (CRP), or rhPTX-2 (SAP), although only one of the Pentraxins may be mentioned in any particular design or example. Also, the fusion partners may be fused to the C-terminus of either PTX-1 or PTX-2 either with or without intervening linkers.

The fusion partners can be any desired protein entity. For example, the fusion partner could be a camelid single-chain antibody binding domain (designated VHH in the literature) designed to bind a viral, bacterial, fungal, or parasite antigen, another infectious agent of interest, or a toxin, or a hapten, or a surface protein or other molecular entity on a cancer cell. In the case of PENTABODIES™ displaying VHH domains: the fusion partner is a selected protein sequence comprising approximately 100-200, 120-180, 130-170, 130-140 amino acids (and increments thereof) in length that binds to the target with high affinity.

Alternatively, in the case of PENTAGENS™, the fusion partner may comprise one or more antigens expressed on the surface of an infectious agent (e.g. viral coat protein, bacterial, fungal, or parasite surface protein). In certain embodiments, such antigens may be modified from their natural state. PENTAGENS™ may be used as vaccines, eliciting an immune response from a patient against the target infectious agent.

Linkers are short sequences of amino acids designed to provide a flexible spacer between the fusion partner and rhPTX-1/2 to allow for unhindered function of both domains. Generally, linkers are less than 30 amino acids. In any particular fusion protein, a linker may or may not be required for optimal function. Typically, several linker designs, including no linker (SEQ ID NO. 3), are tested for their ability to ensure or improve expression, solubility, activity, and/or stability before choosing a final construct for product development. Linkers suitable for use with the current invention are well known to those of skill in the art and include for example, but are not limited to (GGGGS)n (SEQ ID NO: 5), A(EAAAK)nA (SEQ ID NO: 6), Gn, (GSG)n, where n=2-6, KLAAA (SEQ ID NO: 7), KESGSVSSEQLAQFRSLD (SEQ ID NO: 8), and/or EGKSSGSGSESKST (SEQ ID NO: 9).

### DESCRIPTION OF THE DRAWINGS

FIG. 1 provides a schematic crystal structure of hPTX-2 (pdb file: 1SAC). hSAP is a 125 kDa homopentamer comprising 5 identical 25 kDa protomers held together in a disc shape by non-covalent bonds. The diameter of the pentamer is approximately 100 Å with a 20 Å pore in the center and a depth of approximately 35 Å, creating two faces of the pentamer. One face of the molecule, called the ligand-binding face (left side of FIG. 1) contains 10 calcium-binding sites (2 per protomer) through which hPTX-2 binds as an opsonin to a variety of ligands known as danger- and pathogen-associated molecular patterns (DAMPs & PAMPs) to facilitate clearance of foreign particles and cellular debris associated with damaged tissue via the innate immune system. On the opposite face of the pentamer are five Fc receptor binding epitopes (right side of FIG. 1), that bind to Fcg and Fca receptors on monocytes and macrophages of the innate immune system to effect an anti-inflammatory, pro-resolutive biological response to inflammation and fibrosis. The five N- and C-termini of each protomer in the pentamer are indicated by white arrows on the right side of the figure. Native hPTX-2 is glycosylated at Asn32. The rhPTX-1/2 fusion proteins described in this invention are constructed such that the C-terminus of each fusion partner is connected to the N-terminus of the PTX-2 protomer either with or without an optional peptide linker. However, the invention also includes the possibility of C-terminal fusions to the pentraxin chassis. Although PTX-2 structure is the only one displayed here, the PTX-1 structure is similar enough to PTX-2 for the purposes of this invention, and it is assumed that either pentraxin can be used interchangeably in these fusion protein designs, although each pentraxin likely interacts with Fc receptors differently, resulting in distinct downstream FcR-mediated cellular responses that can be exploited for the desired purpose (e.g. vaccine vs. therapeutic) of these fusion proteins. The pentraxin structures enable the ability to modulate binding of the pentraxin proteins to Fc receptor family (e.g. FcgI, II, or III) via site-specific mutations in the FcR epitopes thus “tuning” the immune response. This immune-response tuning represents an added dimension to the invention, increasing the applications of the technology.

FIG. 2 provides a schematic showing a rhPTX-1/2 fusion protein design comprising in this case an N-terminal fusion partner (e.g. including, but not limited to a binding domain, antigen, enzyme, or cytokine) with a peptide linker (top of FIG. 2) or without a peptide linker (bottom of FIG. 2), followed by rhPTX-1/2 at the C-terminal end of the fusion protein (as shown in SEQ ID NOS: 1-4). The necessity for a peptide linker is determined on a case-by-case basis and depends upon the ability of each fusion moiety to preserve its own structure and function as part of the fusion protein, as well as for the fusion protein to be expressed at sufficient levels and to be manufacturable and stable upon storage.

FIG. 3 provides a schematic of a representative PENTABODY™. In an embodiment, the N-terminal fusion partner is a binding domain, such as but not limited to, a camelid single-chain VHH domain, the resulting rhPTX-1/2 fusion protein is known as a PENTABODY™. In this example, a linker between rhPTX-1/2 and the binding domain is shown, but a linker may not be required in all PENTABODY™ designs. A PENTABODY™ is capable of binding up to 5 target antigens or haptens with one molecule, resulting in enhanced binding ability, or avidity, relative to a single binding domain, or two binding domains, as in the case of monoclonal antibodies based on the IgG structure.

FIG. 4 provides a schematic representation of a PENTABODY™ binding to, and crosslinking virus particles in solution. Crosslinking viruses diminishes or eliminates their ability to infect cells, and thereby mitigates a viral infection. Additionally, the native activity of hPTX-1/2 involves facilitating clearance via phagocytosis by innate immune cells of foreign matter from the blood or damaged tissue sites, helping to further diminish a viral infection. Pentraxin-virus complexes internalized by antigen presenting cells may also result in adaptive immune responses, increasing the value of the invention. The pentavalent avidity property of PENTABODIES™ make them very efficient at binding to, crosslinking, inactivating, and clearing target antigens on viruses, other microbial pathogens, or even blood-borne toxins and haptens resulting in very high potency. Alternatively, PENTABODIES™ may crosslink target cell types to facilitate cellular signaling. The capabilities of VHH domains to bind with high affinity to both large and small molecules, and with increased efficiency due to avidity resulting from pentavalent presentation, makes this novel class of therapeutics significantly effective against a wide array of targets.

FIG. 5 provides a schematic showing an alternative embodiment of the novel technology claimed herein, wherein an antigen can be the fusion partner for rhPTX-1/2. This results in a pentavalent presentation of the antigen, called a PENTAGEN™. Due to the manufacturability and stability of the pentraxin chassis coupled with the pentavalent presentation of selected antigens, PENTAGENS™ comprise very potent, effective protein-based vaccines against viruses or other infectious pathogens.

FIG. 6 provides a schematic comprising an example of the technology where the fusion partner is a cytokine, creating a PENTAKINE™. Cytokines are small proteins with highly selective cell-signaling activities, for example, anti-inflammatory activity. Due to the stabilizing properties of the rhPTX-1/2 chassis, PENTAKINES™ can deliver selected cytokine activity with a half-life of approximately 24 hours (based on the plasma half-life of rhPTX-2), longer than most native cytokines. Fusion of a cytokines to the rhPTX-1/2 chassis can be used to normalize the plasma half-life of a variety of cytokines. As one of many possible alternatives, the fusion partner may also be an enzyme or an enzyme inhibitor (not shown).

FIG. 7 provides a schematic representation of heteropentamers possessing intramolecular multivalency. Modern molecular biology technology has enabled the stable insertion of more than one gene into an expression cell line. This technology can be used to introduce from 1-5 distinct rhPTX-1/2 fusion protein coding sequences into a cell line, which may then enable the cell to synthesize and randomly assemble heteropentamers of the target sequences. These heteropentamers presenting from 1-5 distinct fusion partners have the property of intramolecular multivalency, or INTRAVALENCY™. FIG. 7 provides three embodiments of the concept, where the different colors of fusion partners denote distinct sequences and structures: INTRAVALENT™ PENTABODIES™, INTRAVALENT™ PENTAGENS™, and INTRAVALENT™ PENTAKINES™. By presenting multiple binding, antigenic, cytokine, luminescent, toxic, or other activities on one molecule, this novel class of therapeutics enables in highly effective anti-viral, anti-microbial, or anti-cancer therapies, highly effective mono- or multi-valent vaccines, highly potent cytokine cocktails, targeted cytotoxic entities, sensitive diagnostics, or other high-potency biological activities.

FIG. 8 provides a schematic demonstrating intramolecular multifunctionality of rhPTX-1/2 fusion proteins. In addition to heteropentameric fusion proteins comprising one class of fusion partner (i.e. binding domain, antigen, cytokine, or enzyme), cell lines can be constructed to produce multifunctional heteropentamers wherein the fusion partners are mixed classes, for example binding domains and cytokines, toxins, or enzymes. In this way, rhPTX-1/2 fusion proteins can be designed and produced using the binding domains as targeting mechanisms to a particular cell or organ that subsequently delivers the bioactivity of the cytokine, toxin, or enzyme to that local environment. In this way, a selected biological activity can be localized to a target cell, tissue, or organ. For example, these types of rhPTX-1/2 fusion proteins could be used to specifically target cancer cells with the goal of disrupting cancer cell growth and/or metastasis via the activity of the enzyme, internalizing peptide or toxin contained in the targeting pentamer. Additionally, heteropentameric, multifunctional rhPTX-1/2 fusion proteins can be employed as sensitive diagnostic probes for either in vitro or in vivo applications.

FIG. 9 provides a schematic representing one method of producing pentraxin drug conjugates (PDCs). Transfecting cells with more than one Pentraxin fusion protein gene enables production of heteropentameric molecules. In this example, one protamer gene contains a microbial transglutaminase (mTG) glutamine acceptor sequence (Qtag) which can enable mTG-catalyzed site-specific attachment of an amine-containing drug, fluorescent dye, or other small molecule or peptide. The other protamer sequence in this example is a VHH fusion, enabling the heteropentamer to bind to a target antigen, thus potentially targeting the heteropentamer with a drug payload to a target cell, or facilitating detection of a target antigen. A heteropentamer comprising a VHH paired with a drug is a pentraxin drug conjugate (PDC) as shown in FIGS. 9B and 9C, while a VHH paired with a fluorescent dye (FIG. 9A) creates a diagnostic heteropentamer.

FIG. 10 provides a schematic representing Pentagen Vaccine Depot Formulations. Long-lasting depot PENTAGEN™ vaccine formulations can be formed using pentraxin 2 as the pentagen chassis by exploiting the colloidal suspension formation of pentraxin 2 in the presence of calcium ions (Baltz, M., 1982, Biochim et Biophys Acta). Shown in this figure are examples of both monovalent pentagens and multivalent pentagens which are likely to be long lasting and highly immunogenic repeating structural patterns when dosed in a colloidal particulate form. Taken together with the native FcR epitopes on pentraxins with naturally bind to immune cells, these vaccines are likely self-adjuvating, meaning they need no exogenous and potentially toxic adjuvants in order to stimulate potent immune responses. In certain embodiments, the pentagen vaccine depot formulations of the invention comprise PTX2-based pentagens that form colloidal suspensions with Ca+2. They may be intrinsically-adjuvated via Pentraxin FcR binding epitopes in particulate presentation. In certain embodiments, the pentagen vaccine formulations may be designed for subcutaneous administration, or in a format comprising long lasting depot formulation that intensify or prolong immune response by a host. The pentagen vaccines provide the advantage of being available as single dose vaccines.

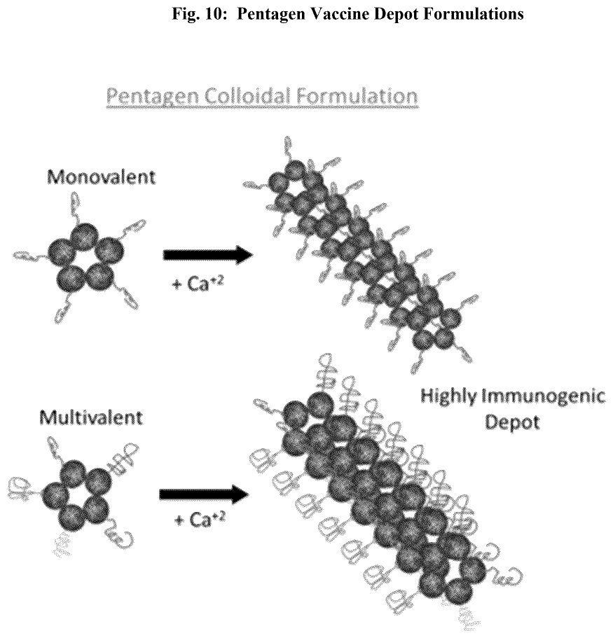

FIG. 11 provides panels representing development and use of HBV diagnostics. A hepatitis B virus (HBV) diagnostic heteropentamer was created on the pentraxin 2 chassis by transfecting cells with a VHH domain shown to be specific for the surface antigen of HBV (HBsAg, VHH5, Serruys, B., 2009, Hepatology 49) along with a PTX2 protomer sequence containing mScarlet fluorescent protein as an N-terminal fusion. In a cell-based assay, HEK293 cells that were transfected with the HBsAg gene were detected after incubation of the HBV heteropentameric diagnostic using the TRITC fluorescence channel (panel B). Cells that were not transfected with HbsAg did not exhibit binding of the diagnostic (panel D), demonstrating specificity of the diagnostic. Panels A and C show the brightfield images of the cells.

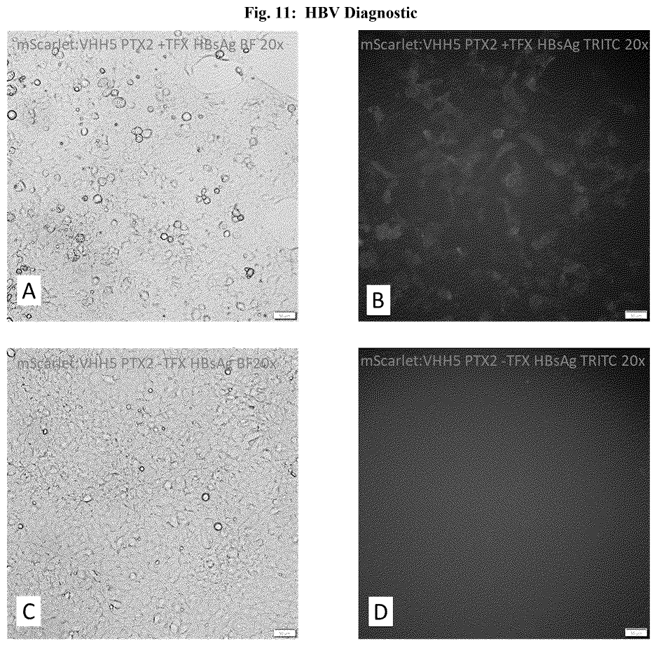

FIGS. 12A and 12B provide the results of an ELISA assay performed to assess the binding specificity of Pentabodies. Pentabodies displaying anti-HBsAg VHH domains (VHH5, Serruys, B., 2009) were shown by ELISA to specifically bind HBsAg with binding constants comparable to a commercial polyclonal anti-HBsAg IgG antibody. Negative controls, non-specific IgG, native PTX-1, and native PTX-2 did not bind to HbsAg.

FIGS. 13A-13E provides the results of an in vitro cell-based HBV inhibition assay, demonstrating that anti-HBV Pentabodies are potent inhibitors of HBV virus. Pentavalent presentation of the VHH5 (Serruys, B., 2009, Hepatology) binding domain on a pentabody resulted in more than a 1000-fold increase in potency relative to the VHH5 domain expressed alone as a monomer. This demonstrates significant synergy via the avidity of pentavalent presentation on a pentraxin chassis.

FIG. 14 provides the results of an ELISA assay demonstrating the activity of anti-SARS-COV-2 pentabodies. Anti-SARS-COV-2 Pentabodies were generated using a VHH domain shown to bind the RBD of SARS-COV-2 spike protein (VHH20, Xiang, Y., 2020, Science). In a competitive ELISA assay, the anti-SARS-COV-2 pentabodies displaying VHH20 were shown to be 4-6× more potent than monomeric VHH20, and more than 50× more potent than a mAb binding to the same RBD region of the spike protein. FIG. 15: Dengue virus-specific diagnostics were created by covalently coupling NHS-activated alexafluor-488 to PTX-2 pentabodies with VHH domains (P2 and DB5, Shriver-Lake L. C., 2018, Nature; Fatima, A., 2014, PLOS ONE) specific to dengue virus NS1 protein (gel on left side of figure). The labeled pentabodies were used to detect NS-1 protein from dengue virus in a flow cytometry experiment. Both pentabodies gave strong signals with FITC means well separated from the negative control (AF-488-labeled PTX-2). These data indicate the feasibility of developing specific and sensitive diagnostics for viral infections.

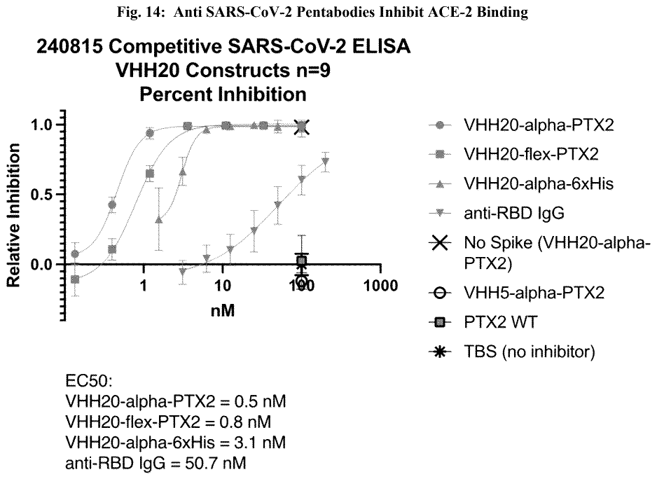

FIGS. 15A-15E provide results and data demonstrating the ability to create a sensitive and specific diagnostic by labeling a pentabody with an Alexafuor dye using NHS chemistry to covalently attach dye to surface lysines. FIG. 15A shows the purification and AF488-labeling of DB5- and P2-Pentabodies by SDS PAGE. FIGS. 15B, C, and D provide flow cytometry data of the negative control (15B, native PTX-2 labeled with AF488, no VHH fusion), AF488-DB5-PTX2 (15C), and AF488-P2-PTX2 (15D). These plots show significant signal with mean FITC signals at least 15× above background as listed in the Table FIG. 15E.

FIGS. 16A and 16B provide, as earlier described in relation to FIG. 9, data and results related to the use of microbial transglutaminase (mTG) to catalyze the conjugation of cadaverine AlexaFluor488 (cAF488) to a heteropentameric PTX2 pentamer comprising at least 80% VHH-20-PTX2 protomers and <20% Qtag PTX2 protomers.

FIGS. 17A and 17B show results and data related to the assessment that as an alternative to covalently coupled molecular payloads on targeting Pentabodies, derivatives of Phosphoethanolamine (PE) can be synthesized and coupled via calcium to PTX fusions to create a delivery vehicle for non-covalently complexed small molecules. This application exploits the natural affinity of PE for PTX proteins in the presence of calcium ions that may facilitate delivery and release of a drug to a target cell. FIG. 17A shows a reaction scheme for covalently coupling NHS-AF488 to PE. FIG. 17B displays a schematic model of amino-derivatized PE coupled via calcium to a pentraxin chassis.

FIG. 18 provides a table showing that as a potential improvement to bispecific or trispecific IgG therapies, Pentraxin fusions can easily be constructed and used for treating cancers and immune system disorders with checkpoint inhibitors and T-cell engagers/stimulators or immune cell modulators. The VHH's and cytokines listed in the table were incorporated into heteropentameric pentabodies in order to demonstrate the utility of this approach. For example, aCD3/aCD20 pentabodies represent a viable alternative to rituximab, a bispecific mAb targeting CD3 and CD20. A pentavalent pentraxin-based version of these binding domains is likely to be more potent than the current IgG therapy. As another example, combining the checkpoint inhibitor aPD-L1 with the T-cell superagonist, IL15N72D on a pentraxin chassis should produce a very potent robust T-cell stimulator for use in cancer therapy. Heteropentameric therapeutics enabled by the present invention are easy to design and manufacture, adding to their value.

### DETAILED DESCRIPTION OF THE INVENTION

It should be understood that this disclosure is not limited to the compositions, methods, or processes contained in this description, as these may expand during product development over time. It is also to be understood the particular terminology used in this description of specific embodiments is not intended to limit the scope of the invention, and that scope of the invention is limited only by the appended claims. As defined otherwise, all technical and scientific terms used herein have the same meaning as commonly understood by one of ordinary skill in the art. Although many methods, materials, compositions, and processes considered equivalent to those described herein can be used in the practice or testing of embodiments of this invention, the currently preferred examples of each are described below. All publications cited in this disclosure are incorporated by reference in their entirety, including United States Patent Application Publication No. 2013/0195861 and United States Patent Application Publication No. 2014/0302024. Nothing herein is to be construed as an admission that this disclosure is not entitled to antedate such disclosure by virtue of prior disclosure.

It must also be noted that as used herein and in the appended claims, the singular forms “a”, “an”, and “the” include plural reference unless the context clearly dictates otherwise. Thus, for example, reference to “pentamer” is a reference to one or more pentamers and equivalents thereof known to those skilled in the art, and so forth.

As used herein, the term “about” means plus or minus 10% of the numerical value of the number with which it is being used. Therefore, about 50% means in the range of 45%-55%.

As used herein, the term “substantially” means being largely but not wholly what is specified. For example, the term “substantially similar” with regard to a nucleotide or protein sequence indicates that the sequence is largely identical to another reported sequence for the same oligonucleotide, protein, or peptide. However, the nucleotide sequence may include any number of variations that do not affect the structure or function of the resulting protein.

As used herein, the term “significantly” means demonstrably different from a comparator without respect to any statistical calculation. For example, the binding affinity of a particular protein variant to a target may be significantly different from another variant without respect to any numerical value.

“Administering” when used in the context of delivering a therapeutic agent means to administer the therapeutic agent to a patient or subject such that the targeted tissue, organ, or system is positively impacted by the therapeutic agent. For example, the therapeutic agent may be a composition containing an rhPTX-1/2 fusion protein designed to bind and clear a virus from the blood compartment of a patient or subject when administered intravenously. Thus, as used herein, the term “administering” can include, but is not limited to, systemic or local delivery of an rhPTX-1/2 fusion protein via intravenous injection, subcutaneous injection, intramuscular injection, intranasal inhalation, oral inhalation into the lungs, or topical application whereby the therapeutic agent reaches the target tissue, organ, or system. Such administration can be facilitated by combination with other techniques such as heating, radiation, ultrasound, and the use of other delivery agents or devices, for example transdermal patches, implanted injection devices, dry powder inhalers, or nebulizers.

“Providing”, when used in conjunction with a therapeutic, means to administer a therapeutic directly into or onto a target tissue, or to administer a therapeutic to a patient whereby the therapeutic agent positively impacts the tissue to which it is targeted.

The term “animal” as used herein includes, but is not limited to, humans, and non-human vertebrates such as wild, domestic, laboratory, outbred and inbred species, and farm animals.

The term “improves” is used to convey that the present invention changes either the characteristics and/or the physical attributes of the tissue to which it is provided, applied, or administered. The term “improves” may be used in conjunction with a diseased state such that when a diseased state is “improved”, the symptoms or physical characteristics associated with the diseased state are diminished, mitigated, or eliminated.

The term “inhibiting” generally refers to prevention of the onset of the symptoms, alleviating the symptoms, or eliminating the disease, condition or disorder. The term “inhibiting” can also refer to a molecular activity whereby one molecule inhibits the natural bioactivity of another molecule, rendering the inhibited molecule inactivated. For example, binding of an rhPTX-1/2 fusion protein to a target molecule may inhibit the activity of the target molecule. “Inhibition” may also refer to a biological pathway or progression. For example, binding of an rhPTX-1/2 fusion protein to a target virus can inhibit the virus' ability to infect cells.

The term “fusion partner” signifies a moiety, such as a protein moiety, fused to the N-terminus of rhPTX-1/2; the term includes the protein sequence, and hence structure and function, of the moiety encoded for in a DNA strand and synthesized as one transcript to create a single protein chain. Such fusion partners may or may not be linked to an intervening protein sequence known as a linker peptide. Linkers are used to provide structural space between fusion partners in order to ensure proper structure and function of multiple partners, and to maximize stability of the resulting fusion proteins. The term “chassis” refers to the rhPTX-1/2 pentameric core of an rhPTX-1/2 fusion protein. The term “display” is used to describe the structural arrangement of the N-terminal fusion partners oriented in a radially-symmetrical pattern extending outward from the outer perimeter of the rhPTX-1/2 chassis, or generally the arrangement of N-terminal fusion partners that are structurally distinct from the chassis, and solvent accessible to facilitate intermolecular interactions with target molecules.

“Optional” means that the subsequently described event or circumstance may or may not occur, and the description includes instances where the condition occurs, and instances where it does not. For example, the linker between a fusion partner and the rhPTX-1/2 moieties of the fusion protein may be optional, depending on each particular case.

Throughout the application, various terms may be used such as “primary”, “secondary”, “first”, “second”, and so on. These terms are words of convenience in order to distinguish between different elements, and such terms are not intended to be limiting as to how the different elements may be utilized.

Also used herein, “isolated”, means altered or removed from the natural state through human intervention. For example, hPTX-1/2 circulating in human blood is not “isolated”, whereas purified rhPTX-1/2 fusion proteins have been isolated from the cell culture milieu in which they were synthesized.

The terms “mimetic”, “peptide mimetic”, and “peptidomimetic” are used interchangeably herein, and generally refer to a peptide, partial peptide, or non-peptide molecule that mimics the structure and function of a selected native peptide. These peptide mimetics include recombinantly or chemically modified peptides, and synthetic polymeric non-peptides. For example, the linkers between the fusion partners and rhPTX-1/2 moieties in rhPTX-1/2 fusion proteins may be natural peptides or peptide mimetics.

The terms “pharmacologically acceptable”, and “physiologically tolerable” including grammatical variations thereof used in the context of administering a therapeutic agent refer to the ability to administer compositions, carriers, diluents, excipients, as well as other non-pharmacologically active ingredients in a formulation that do not elicit undesirable physiological effects after dosing to the patient or subject.

As used herein, the terms “therapeutic”, “therapeutic agent”, or “therapeutic protein” means an agent used to treat, combat, ameliorate, mitigate, prevent, or improve an unwanted condition or disease of a patient. In part, the embodiments of the present invention are directed toward treating viral or microbial infectious diseases, either as treatments for active infections or as vaccines to elicit an immune response in order to prevent or mitigate infections, to inhibit and clear blood-borne toxins or other pathogens, and to modulate cytokine imbalances. The activity contemplated herein includes both medical therapeutic intervention, and/or prophylactic treatment, as appropriate.

The terms “therapeutically effective”, “therapeutically effective dose”, “effective”, or “effective dose” as used herein, may be used interchangeably and refer to an amount of a therapeutic composition comprising embodiments of the present invention necessary to effect the desired therapeutic response in a patient or subject. The required therapeutic amount may be calculated or otherwise derived from pre-clinical experimentation, or from human clinical trials. The exact dosing and dosing regimen will be dependent on the disease being treated, the dosing route and schedule, and the plasma half-life and potency of each individual therapeutic agent.

The terms “treat”, “treated”, or “treating” are used herein to refer to both therapeutic and prophylactic or preventative measures, wherein the objective is to prevent, mitigate, or produce a beneficial outcome for a disease state resulting in the improvement of a patient's or subject's health. For the purposes of this disclosure, beneficial or desired clinical outcomes include, but are not limited to, alleviation of symptoms, diminishment of the extent of the condition, disorder or disease; stabilization (i.e. not worsening) of the state of the disease, delay or slowing of the onset or progression of the disease, amelioration of the condition. Treatment includes eliciting a clinically significant response without excessive unwanted side effects. Treatment may also include prolonging survival as compared to expected survival if not treated.

The term “tissue” refers to any aggregation of similarly specialized cells which are united in the performance of a particular function. An “organ” is a structurally organized collection of tissues performing specific functions to maintain the health of a patient or subject.

As used herein, the terms “antibody”, “antibodies”, “antibody fragment”, “antibody binding domain”, “binding domain” as well as terms associated with antibody structure such as single-chain variable fragment, “scFv”, shall extend to all antibodies, antibody fragments, and antibody-like binding domains, including, but not limited to, VHH proteins, and annexins.

Human Pentraxin-2 (hPTX-2), is constitutively expressed in the liver and circulates at a constant level in the blood at approximately 30 μg/mL. In contrast, hPTX-1 is an acute-phase protein normally present at very low levels (approximately 1 μg/mL) in human serum, with expression levels transiently increased up to 1000-fold in response to inflammatory events (Gewurz H, et al, 1982, Adv Intern Med., 27:345-72. PMID: 7041546). Human PTX-1/2 is a non-covalent homopentamer comprising 5 protomers individually synthesized and subsequently assembled by the cell into a disc shaped pentamer prior to secretion into the extracellular space. Human PTX-1/2 functions as part of the innate immune system to resist microbial infections and to scavenge and clear cellular debris from damaged tissue. Additionally, hPTX-2 appears to play a role in mitigating inflammatory cell signaling and fibrosis whereas hPTX-1 appears to respond to inflammatory signals. These functions of hPTX-1/2 involve (i) Ca+2-dependent binding to ligands associated with microbes (pathogen associated molecular patterns, or PAMPs) and cellular debris (danger associated molecular patterns, or DAMPs) via calcium ions bound to one face of the pentamer (ii) facilitating complement response and opsonization by C3b and iC3b, (iii) binding to Fcg or Fcα receptors on monocytes and macrophages via FcR binding epitopes on the opposite face of the pentamer to directly opsonize and promote phagocytosis or these pathogenic materials, and (iv) subsequent regulation of monocyte phenotypes away from pro-inflammatory and pro-fibrotic signaling and towards anti-inflammatory, pro-resolutive phenotypes. Conversely, depending on which pentraxin chassis (PTX-1 vs PTX-2) forms the core of the fusion protein, the FcR-mediated signaling may result in pro-inflammatory signaling resulting from the nature of interaction with the FcR. By virtue of the distinct binding and regulatory/cell signaling functions of hPTX-1/2, its activity is localized to sites of damaged tissue and repair.

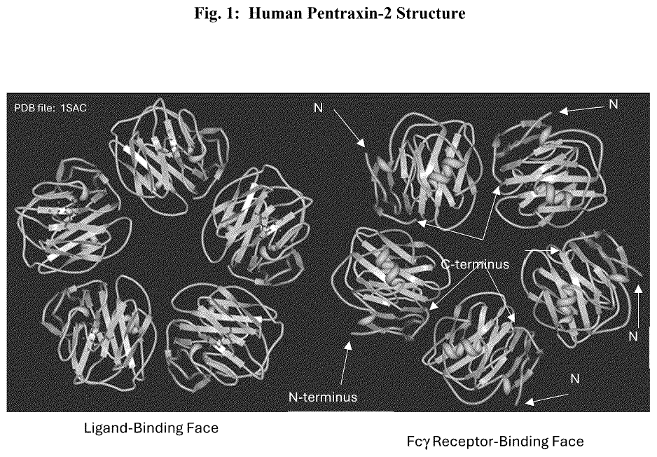

Both the N- and C-termini of each hPTX-1/2 protomer are accessible to solvent, and do not appear to be involved in subunit interactions. The N-terminus of each protomer extends outward radially from the surface of the pentamer, while the C-termini are located in the interior area of the pentamer, near the central pore, directed in a perpendicular direction away from the plane of the pentamer. The pentamer is held together by extensive inter-protomer interactions involving mostly van der Waals interactions within an extensive hydrophobic patch and including two buried salt bridges. Collectively, these interactions account for the considerable stability of the pentamer (FIG. 1).

Human Pentraxin-2 is a member of the short pentraxin family, along with human pentraxin-1, also known as C-reactive protein, or CRP. In contrast, hPTX-3, among others, is known as a long pentraxin. Long pentraxins exhibit an N-terminal extension relative to short pentraxins, 160 amino acids in the case of hPTX-3, demonstrating the feasibility of creating N-terminal fusion proteins from short pentraxins, in particular hPTX-1/2, resulting in distinct functional domains that preserve the functions of both the fusion partner and the core hPTX-1/2 structure in rhPTX-1/2 fusion proteins.

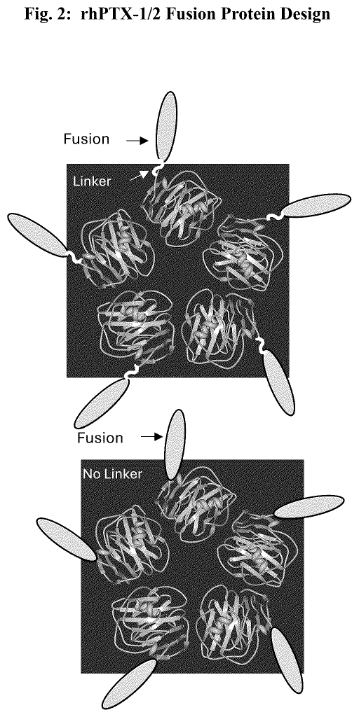

Also, because hPTX-1/2 is a naturally formed non-covalent pentamer, N-terminal rhPTX-1/2 fusion proteins have the capacity to present or display 5 fusion partner domains to the outside perimeter of the fusion protein (FIG. 2). If those fusion partner domains are binding proteins, or antibody structures, such as V_{H}H proteins from camelids, then the potential exists for enhanced binding affinity, known as avidity, resulting from pentavalent presentation of the binding domains. This accumulated strength of binding conferred by multiple interactions in conjunction with the 5-fold rotational symmetry of the pentamer can create a very effective class of binding molecules capable of tightly crosslinking and thereby inactivating target antigens, for example on viral, microbial, fungal, or parasite surfaces. Combining the properties of selected fusion partners (e.g. binding domains, antigens, enzymes, cytokines) with the properties inherent in hPTX-1/2 provides a novel class of highly effective therapeutics, and vaccines. Alternatively, the binding properties of the hPTX-1/2 chassis, or a binding domain fusion partner localizing to damaged tissue or a selected target can be used to create diagnostic agents via rhPTX-1/2 fusion proteins, for instance, enabling the creation of targeted imaging agents.

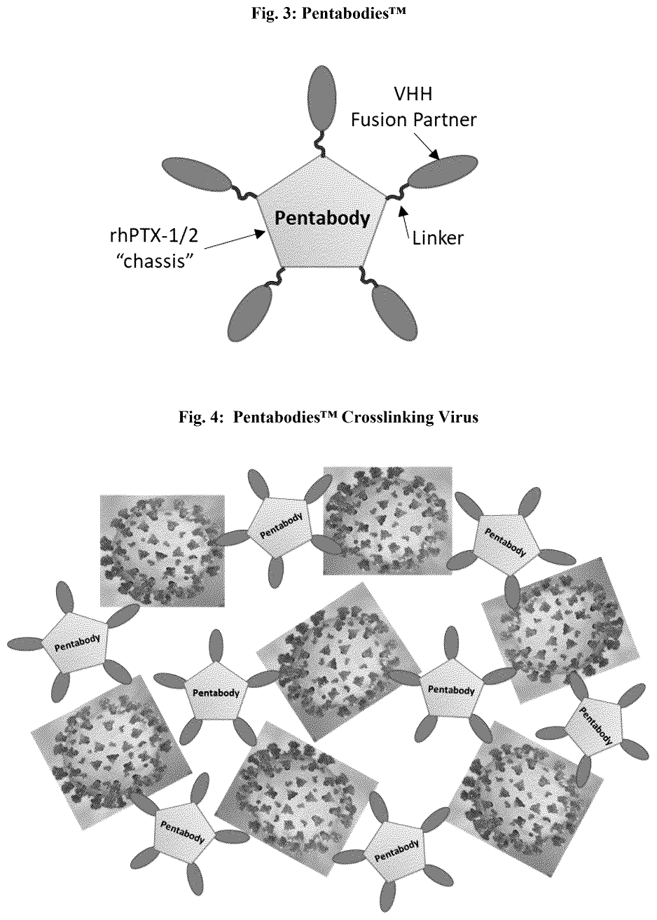

Embodiments of this invention are generally directed to the use of hPTX-1/2 as a chassis for pentavalent presentation of a selected biological component or activity, for example a binding domain for use as a therapeutic, an antigen for use as a vaccine, or a cytokine for use in modulating cytokine balances influencing cell populations. This invention can also be used to deliver pentavalent presentations of other biological activities inherent in proteins, including, but not limited to, enzymatic activity, signaling or internalization peptides, hormones, inhibitors, agonists, or antagonists. Other embodiments are directed to pharmaceutical compositions that include rhPTX-1/2 fusion proteins, and methods of using those compositions. Other embodiments are directed towards methods of making rhPTX-1/2 fusion proteins, either as homopentamers, or as heteropentamers (FIGS. 3-8).

An rhPTX-1/2 fusion protein as defined herein is a molecule exhibiting combined structural and functional properties of both rhPTX-1/2, and one or more selected fusion partner(s). The fusion partners may be of a single type, or of multiple types. In this way, rhPTX-1/2 serves as a chassis upon which are covalently attached from 1-5 distinct protein domains of selected function. The rhPTX-1/2 chassis or hub serves to orient the fusion partners in a radially symmetrical fashion much like spokes of a wheel, maximizing accessibility to the target and providing the potential for avidity.

While the preferred embodiment uses molecular biological techniques to construct fusion protein protomers from a single DNA transcript with an optional peptide linker between the fusion partner and rhPTX-1/2, the linkers may contain unnatural amino acids or be synthetic molecules able to chemically link C-terminus of the fusion partner with N-terminus of rhPTX-1/2. As would be evident to those skilled in the art, modified, or synthetic linkers can create stable covalent bonds between the fusion partners, or they may be designed to hydrolyze after a period of time post-dosing so as to release the fusion partners into solution, depending on the application.

While this invention specifies human pentraxin-1/2 in certain embodiments, other embodiments include pentraxin-1/2 from other animal sources, in addition to variants of hPTX-1/2. Such variants may include modifications of single or multiple amino acids by site-directed mutagenesis, or by chemical modification of targeted amino acids while preserving the core pentameric structure and function of the PTX-1/2 moiety of the fusion protein.

In some embodiments, the rhPTX-1/2 protomer may be 100% identical to native hPTX-1/2 (SEQ ID NOs. 1 & 2). In other embodiments, the PTX-1/2 protomer may have an amino acid sequence that is at least 60%, at least 70%, at least 80%, at least 85%, at least 90%, at least 95%, at least 97%, at least 98%, at least 99% identical to SEQ ID NOs. 1 & 2. In some embodiments, the amino acid sequence may be altered to improve manufacturability, potency, or stability, or to modulate the biological activity of the resulting fusion protein. In some embodiments, the amino acid substitutions may be considered conservative in that they preserve as much as possible either the structural or chemical properties of the replaced amino acid. In other embodiments, the amino acid substitutions may confer different structural or chemical properties to the fusion protein while preserving the core pentameric structure of the fusion protein. In some embodiments, the replacement amino acid may not be one of the 20 naturally occurring amino acids normally found in proteins, but rather a non-natural amino acid incorporated into the rhPTX-1/2 fusion protein to confer a desired chemical or structural characteristic.

In some embodiments, there may be a desire to modify and/or customize structural features of rhPTX-1/2 protomers that confer specific functions, such as Ca+2 binding, FcgR or FcaR binding, or glycosylation. In certain cases, it may be advantageous via site-directed mutagenesis to alter, increase, or eliminate any or all these structural features of rhPTX-1/2, depending on the purpose of the rhPTX-1/2 fusion protein. For example, it may be advantageous to remove the heterogeneity inherent in glycoprotein manufacturing by removing the consensus sequence enabling glycosylation at position 32 of rhPTX-2, resulting in a protein that is more streamlined to manufacture while preserving the core functions of rhPTX-2. In another example, it may become advantageous via site-directed mutagenesis to modify the ability of the pentamer to bind Ca+2, thus eliminating or enhancing its ability to bind to ligands via Ca+2 in order to optimize the intended function of the rhPTX-1/2 fusion protein. In other embodiments, disulfide bonds via pairs of cysteines may be introduced by site-directed mutagenesis into the fusion protein structure to aid in stabilizing a desired structure for optimal function. In still other embodiments, it may be advantageous to alter the FcR affinity of the rhPTX1/2 pentamer via site directed mutagenesis of the protomer to modulate the effector functions of these fusion proteins. In certain cases, it may be desired to engineer FcR binding epitopes to increase binding to FcRn, resulting in significantly longer plasma half-life of the molecule.

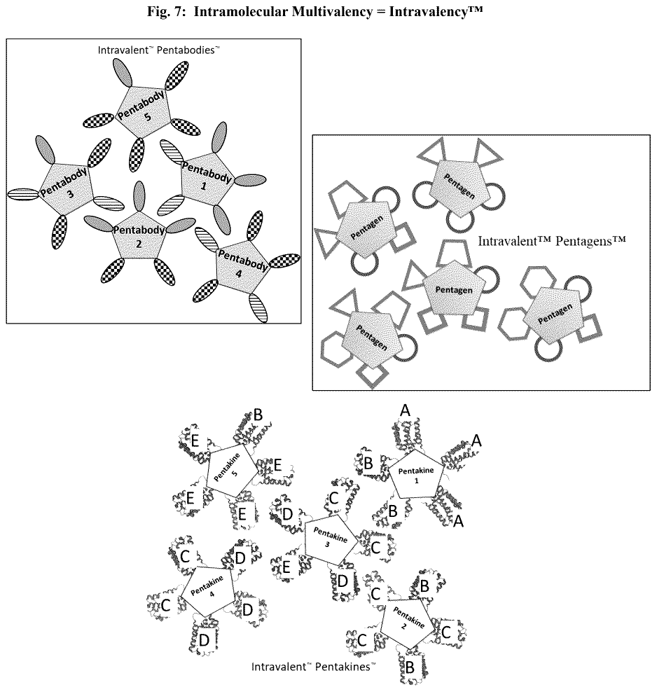

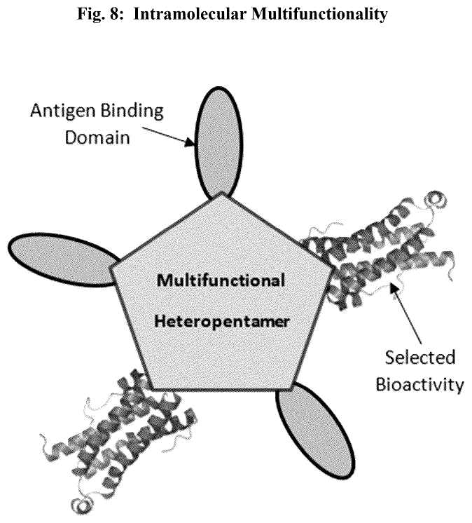

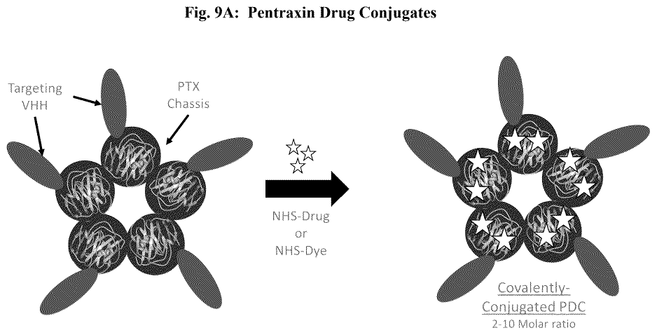

In some embodiments, all 5 protomers will contain identical protein sequences, resulting in a homopentameric fusion protein by virtue of the rhPTX-1/2 pentameric chassis. In other embodiments, from 1-5 distinct protomers may be expressed in varying ratios by the production cell line, resulting in heteropentameric fusion proteins using molecular biological techniques such as those described in the literature (for example, but not limited to, production in CHO cells: Baser, B. et al, 2016, Methods 95, pp 3-12; or Gaidukov, L, et al, 2018, Nucleic Acids Research, Vol 46, No. 8, pp 4072-4086). Using these cell engineering technologies can create production cell lines that produce heteropentameric fusion proteins with defined ratios of distinct pentamers so that two, three, four, or five different fusion partners may be displayed on one molecule using the same rhPTX-1/2 chassis, thus conserving the structure and function of pentameric core derived from rhPTX-1/2 (FIG. 7). This results in intramolecular multivalency, or INTRAVALENCY™. Additionally, this technology also provides a way to incorporate native rhPTX-1/2 protomers into a heteropentamer so that from 1-5 protomers comprising the pentamer may be fusion proteins or native rhPTX-1/2 protomers (SEQ ID NOs. 1 & 2) with no N-terminal extensions or linkers. Incorporating native rhPTX-1/2 protomers into the heteropentamers affords an additional level of control over the structural and therefore functional characteristics of the heteropentamer. For example, in one application, it may be preferable to create an rhPTX-1 fusion protein, whereas in another application, it may be preferable to create an rhPTX-2 fusion protein due to the inherent biological activity differences of these two Pentraxins. This technology can be used to produce any rhPTX-1/2 fusion protein, including, but not limited to, display of binding domains known as PENTABODIES™ (FIG. 3), display of antigens known as PENTAGENS™ (FIG. 5), or display of cytokines, known as PENTAKINES™ (FIG. 6).

In some embodiments, rhPTX-1/2 heteropentameric fusion proteins can be designed and produced with more than one class of fusion partner (i.e. binding domain, antigen, cytokine, enzyme). For example, a fusion protein might contain one, two, three, or four binding domain fusion protomers, in conjunction with four, three, two, or one cytokine or enzyme fusion protomers. In this way, the binding domains can be used to target the heteropentamer to a desired cell, tissue, or organ to subsequently deliver the bioactivity of the cytokine, enzyme, or selected fusion partner. These rhPTX-1/2 pentamers possess intramolecular multifunctionality that may be useful therapeutic or diagnostic agents.

The production cell lines for rhPTX-1/2 fusion proteins may also utilize other eukaryotic (e.g. HEK293, PerC6) and prokaryotic hosts (E. coli). Expression in E. coli may result in formation of insoluble inclusion bodies that require refolding in order to obtain native protein structure and function. Refolding proteins from inclusion bodies is a standard technique practiced by those skilled in the art of recombinant protein production. In the case of refolding rhPTX-1/2 fusion proteins, this may offer an advantage to producing non-glycosylated homo-, or hetero-pentameric fusion proteins at lower cost relative to eukaryotic systems due to lower manufacturing costs of microbial vs mammalian production platforms.

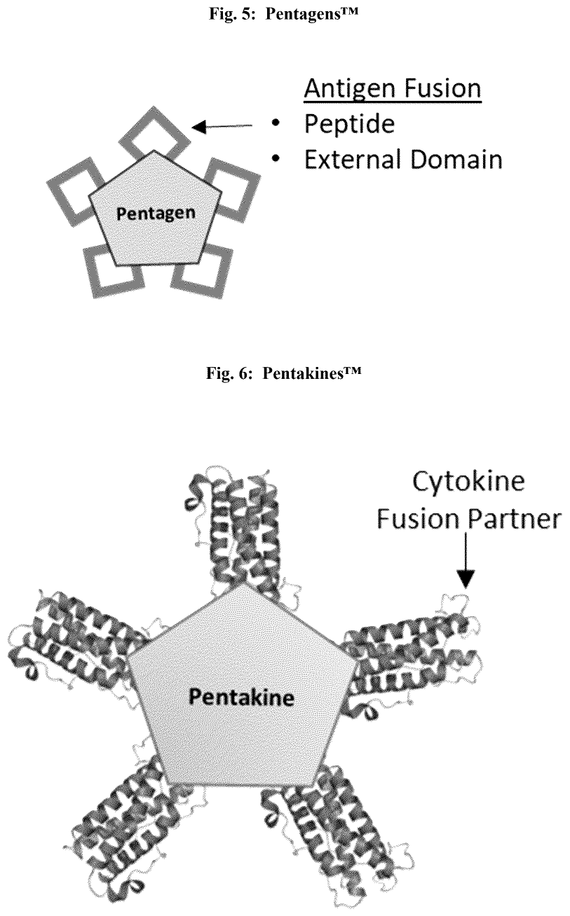

In certain aspects, the invention provides rhPTX-1/2 fusion proteins that bind to a viral protein, such as the spike protein in SARS-COV-2, or selected coat proteins of any virus of interest, including but not limited to, MERS coronavirus, RSV, influenza, parainfluenza, Ebola, HIV, hepatitis B, hepatitis C, viral meningitis, yellow fever, human papilloma, polio (FIG. 4), or any virus that presents a health threat. Alternatively, rhPTX-1/2 fusion proteins can be designed to bind to and inactivate any of the many bacterial pathogens including, but not limited to, Vibrio cholerae, Streptococcus pneumonia, Neisseria mengitidis, Mycobacterium tuberculosis, Clostridioides difficile, including any drug-resistant microbial pathogens such as Acinetobacter, Candida suris, Neisseria gonorrhoeae, Candida, enterococci, and Campylobacter. In certain embodiments, rhPTX-1.2 fusion proteins can be designed to bind to target fungal proteins present in fungal infections including but not limited to fungal diseases such as Aspergillosis, Blastomycosis, Candidiasis, and Pneumocystis pneumonia. In still other aspects, the fusion protein binding domains can be targeted to antigens found in parasites such as, but not limited to, Malaria, Schistosomiasis, Leishmaniasis, African trypanosomiasis, or Giardiasis. In certain aspects, the binding domains on rhPTX-1/2 fusion proteins may be designed to bind and crosslink target cell populations in order to facilitate cellular signaling amongst a cell population.

Any rhPTX-1/2 fusion protein may be formulated in a pharmaceutical preparation comprising the rhPTX-1/2 fusion protein and a pharmaceutically acceptable carrier or excipient. These formulations may also include one or more additional compounds that are pharmacologically active or not, depending on the disease to be treated, for instance an anti-viral such as remdesivir, or an antibiotic such as vancomycin.

Fusion proteins of rhPTX-1/2 may be formulated in a number of ways, depending on the indication and the most effective route of administration. In some embodiments, the rhPTX-1/2 fusion protein may be formulated for intravenous delivery. In other embodiments, the formulation may be intended for intramuscular or subcutaneous delivery, and may contain an adjuvant if the product is intended to be used as a vaccine in order to facilitate a robust immune response. For example, a PENTAGEN™ vaccine may be formulated as a calcium complex with Phosphoethanolamine-derivatized microbeads acting as an adjuvant to present an immobilized surface of antigen to the immune system for a more robust immune response. In other embodiments, the rhPTX-1/2 fusion protein may be contained in a spray dried formulation for use with a dry powder inhaler for local delivery to the upper respiratory tract including the nasal mucosa and/or lower respiratory tract, including the lung. In other embodiments, the formulation may be a liquid intended for delivery to the upper respiratory tract and/or lung with a nebulizer. In other embodiments, an rhPTX-1/2 fusion protein may be formulated for intranasal delivery by a mist or atomized spray. In other embodiments, the rhPTX-1/2 fusion protein may be contained in a liposome formulation with the appropriate lipid, excipient and stabilizer composition to ensure delivery and exposure to the target cells.

Embodiments of the invention also include methods for preparing rhPTX-1/2 fusion proteins. In some embodiments, a DNA sequence coding for a fusion partner (e.g, camelid V_{H}H domain, antigen, cytokine, enzyme, inhibitor, agonist, antagonist, etc) can be added using standard molecular biological techniques to the 5′ end of the fusion protein coding sequence with or without an intervening peptide linker sequence at the 3′ end of the fusion partner coding sequence followed by the rhPTX-1/2 DNA coding sequence such that the N-terminus of the resulting rhPTX-1/2 fusion protein begins with the fusion partner, followed by the peptide linker (or not followed if no linker is necessary for function), which is then followed by rhPTX-1/2 at the C-terminus of the fusion protein. This fusion protein coding sequence can be inserted appropriately into an expression plasmid or vector using standard techniques and then introduced into an expression cell line for production of the protein. Alternatively, the fusion protein coding sequence can be inserted into the genomic DNA of a cellular host (e.g. Chinese Hamster Ovary cells, or CHO cells) to produce a stable production cell line. In some embodiments, targeted integration of the open reading frame encoding the rhPTX-1/2 fusion protein into predetermined loci within the genomic CHO DNA can be used to insert one or more copies of either the same or different rhPTX-1/2 fusion proteins to create either homo- or heteropentamers, depending on the application as described above.

The expression vectors of various embodiments may include any number of additional sequences necessary for promotion, replication, selection, transcription, and the like, and any of the numerous such sequences known in the art may be used. For example, varying gene copy numbers or using different promoters at different genomic integration sites can be used to control expression of selected different rhPTX-1/2 protomers, resulting in a desired and controlled composition of a heteropentamer. These promoters may be constitutive or inducible, depending on the need at hand.

The expression systems may vary between certain embodiments and can include, but are not limited to, bacterial, yeast, insect, or mammalian cells, including human cells. This includes the use of a gene therapy or mRNA format wherein the rhPTX-1/2 protein is expressed in situ in target human patient cells following administration of a gene therapy product capable of causing cells in a patient's or subject's body to express rhPTX-1/2 fusion proteins.

Following expression in a suitable host cell, pentameric rhPTX-1/2 fusion proteins may be isolated and purified using known techniques such as centrifugation, filtration, column chromatography, viral inactivation and removal, concentration and diafiltration, and manipulation of pH, conductivity, and temperature. In certain embodiments, where the rhPTX-1/2 protomers are expressed as insoluble inclusion bodies, pentameric rhPTX-1/2 fusion proteins may be isolated and purified as above after they are first solubilized and refolded into their native state by known techniques for refolding proteins. In the case of rhPTX-1/2 fusion protein pentamers formed with refolded proteins, control of each protomer in the pentamer can be achieved by varying the relative amounts of each desired protomer in the refolding operation resulting in the desired composition of each protomer in the pentamer.

Once purified, rhPTX-1/2 fusion proteins can be formulated into a pharmaceutically acceptable dosage form intended to enable the chosen delivery route. Delivery routes are chosen on the basis of the clinical indication and/or target exposure. Examples of delivery routes include, but are not limited to, intravenous injection, subcutaneous injection, intramuscular injection, intravitreal injection, inhalation through the nose or oral cavity, or topical application to the eye, dermis, lung, or mucosal tissues. For example, an rhPTX-1/2 fusion protein configured as a vaccine (i.e. PENTAGEN™) can be formulated as nanoparticles (for example, by spray drying) delivered by nasal inhalation for delivery to the nasal mucosa of the upper respiratory tract where the initial infection from a number or respiratory viruses occurs (e.g. influenza, RSV, parainfluenza, adenovirus, coronaviruses) (Al-Halifa, S, et al 2019, Frontiers in Immunology 10:22). Delivery to the upper respiratory mucosa is able to induce an IgA response first that protects mainly the upper respiratory tract where respiratory viruses enter the body, followed by an IgG response later which mainly protects the lower respiratory tract (Krammer, F, 22 Oct. 2020, Nature 586, pp. 516-527).

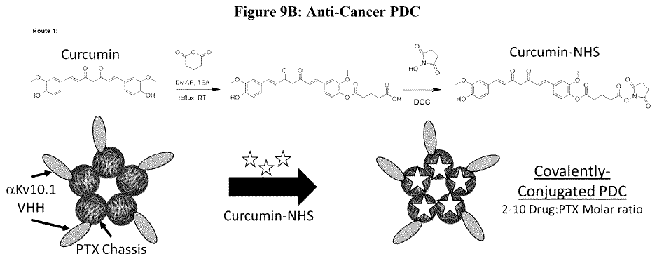

The rhPTX-1/2 fusion proteins made as described herein may be used for therapeutic, prophylactic, or diagnostic (both in vivo and in vitro) applications. Pentameric presentation of fusion partners may result in avidity, or enhanced specificity due to combined strength of multiple bond affinities. This may result in higher-potency, lower-toxicity molecules relative to those presenting only one or two binding or biologically-active moieties. As shown in FIG. 8, heteropentameric rhPTX-1/2 fusion proteins can be used to target biological activity (e.g. cytokine, inhibitor, agonist, or enzyme) to a desired target cell or receptor in vivo or in vitro via one or more protomers exhibiting a specific binding domain. In addition, the rhPTX-1/2 core can also be used to non-specifically target damaged tissue, apoptotic cells, or pathogen-associated molecular patterns as that binding capacity is intrinsic to native hPTX-1/2. As illustrated in FIG. 4, the unique spatial pentavalent arrangement of binding domains presented by an rhPTX-1/2 fusion protein may result in very efficient crosslinking of target cells, viral particles, or microbial pathogens. Bringing cells into close proximity to facilitate multiple interactions may lead to desired biological effects as observed for macrophages, monocytes, neutrophils, T cells, B cells, dendritic cells which all interact with each other. Crosslinking viruses or other infectious pathogens may effectively neutralize or inactivate them, preventing them from infecting more cells, and the activity inherent in PTX-1/2 may then help clear crosslinked viruses from the blood via phagocytosis.

In certain embodiments, rhPTX-1/2 fusion proteins can be used to bind other molecular targets to neutralize their activity. In particular, VHH domains have been shown to exhibit greater structural diversity and greater versatility at binding antigens than those from IgG VL/VH binding domains (Hoey, R J, et al, 2019, Experimental Biol, and Med., 244:1568-1576). Additionally, VHH domains have been shown to bind tightly to small molecule haptens (Ding, L, et al, 2019 Febs Letters, 593:1248-1256) whereas VH/VL domains from IgGs are not as effective. Therefore, it is possible that rhPTX-1/2 fusion proteins displaying engineered VHH domains, especially in a pentavalent manner, may more effectively be used to bind, inactivate, and clear exogenous or endogenous small molecule toxins or haptens, including excessive levels of endogenous molecules like cytokines or hormones.

Pharmaceutical compositions of this invention may also be administered to any animal, particularly any animal that may experience benefit following administration of an rhPTX-1/2 fusion protein, including, but not limited to, humans, canines, felines, livestock, horses, cattle, sheep, poultry, and the like. The required doses, dose regimens, routes of administration, and specific formulations of the rhPTX-1/2 protein will be dependent on the particular animal being treated, and the particular indication being addressed.

Although the present invention has been described in considerable detail with reference to certain preferred embodiments thereof, other versions are possible. Therefore, the spirit and scope of the appended claims should not be limited to the description of the preferred version contained within this specification.

In order that the subject matter disclosed herein may be more efficiently understood, examples are provided below. It should be understood that these examples are for illustrative purposes only and are not to be construed as limiting the claimed subject matter in any manner.

### Example 1

### Pentraxin Drug Conjugates

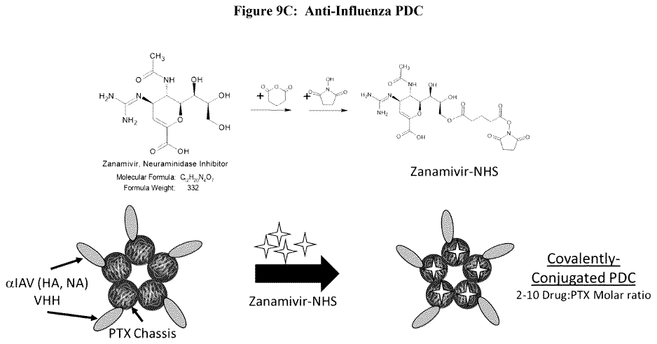

As shown in FIG. 9, pentabodies can be conjugated to dyes and or drugs to create diagnostics or pentraxin drug conjugates (PDCs). In certain embodiments, activated NHS esters can conjugate payloads via PTX surface lysines (FIG. 9A).

In certain embodiments, PDCs comprising curcumin may be created via derivatization of curcumin with NHS (see FIG. 9B). Kv10.1 is a K-channel and is expressed on cancer cells. Curcumin is known to induce apoptosis in cancer cells while remaining non-toxic to healthy cells. A schematic demonstrating the making of such a PDC is provided in FIG. 9B.

In certain embodiments, PDCs comprising Zanamivir may be created following the schematic diagram provided in FIG. 9C.

Zanamivir, also known as CD388 is a drug-FC conjugate. It is a neuramindase inhibitor and has no direct IAV targeting activity. Zanamivir can be derivatized to create an NHS-ester form that can covalently couple to surface lysines on proteins. Coupling Zanamivir to an influenza-targeting Pentabody represents a potential improvement to current therapies by enabling targeting of the drug to the virus or cells infected with virus. This coupling represents the ability of utilizing the novel pentraxin structures provided herein to deliver antiviral activity directly to an targeted (infectious) virus. Pentameric presentation results in increased potency via avidity. Furthermore, exploiting molecular specificity of VHH domains in pentabodies generates precise targeting activity: «IAV, H5N1, NA or HA. In addition, FcR binding epitopes on PTX chassis may generate innate/adaptive immune crosstalk via antigen presenting cells (APCs). This represents the potential to generate specific immune response to targeted viruses.

### Example 2

### Anti-HBsAg Pentabodies Specifically Bind HBsAg

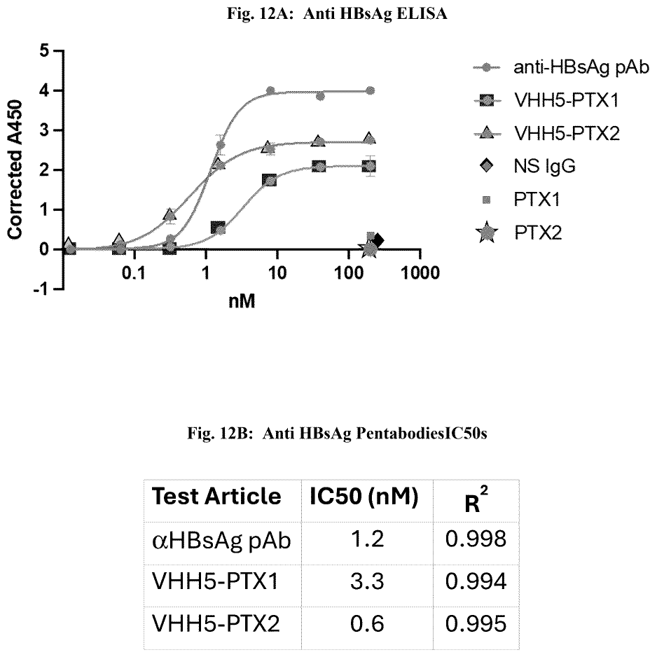

FIGS. 12A and 12B demonstrate the specificity and potency of anti HBsAg Pentabodies in an ELISA format. VHH5-PTX2 Pentabodies on both the PTX1 and PTX2 chassis as well as a control polyclonal Ab specific to HBsAg bind specifically to plates coated with HBsAg protein with IC50 values <5 nM. A control IgG (not specific to HBsAg) and PTX-1 and PTX-2 native pentraxins showed no signal at 200 nM in this ELISA, confirming potent targeting activity of VHH5.

### Materials:

i. Pentraxin variants were expressed in ExpiCHO cells by transient transfection of pcDNA3.4-based vectors according to manufacturer's instructions, and purified to >95% purity by Phosphoethanolamine (PE) affinity chromatography.
ii. Xpressbio HBsAg-coated ELISA plate, Cat #WB2396.
iii. Anti-HBsAg mouse pAb (Fitzgerald Industries, cat #10-H05H

Competitive ELISA Method Summary: 50 μL of 2× test article were added to wells in duplicate. 50 μL of HBsAg-HRP conjugate were immediately added to each well. Plates were inclubated at 37 C for 1 h and developed per manufacturer's protocol.

### Example 3

### Anti-HBsAg Pentabodies are Potent Inhibitors of HBV

Method: This assay measures the inhibition of HBV infection in human liver cells. HepG2-NTCP-C4 cells were seeded onto a collagen-coated 96-well plate. The next day cells were pre-treated with culture media containing 2% DMSO. Cells were infected with 5 uL/well 2024 WT HBV 24 h later, and simultaneously treated with 3-fold dilutions of either VHH5-Pentraxin Pentabodies, monomeric VHH5 (VHH5 is specific for HBsAg), or a mAb specific for HBsAg, The VHH5-containing test articles were tested starting at 5 nM, in duplicate. The Anti-S antigen monoclonal antibody was tested starting at 50 nM. HBV eAg CLIA and Cell Titer Glo (cell viability) assays were performed at 5 days post infection (dpi).

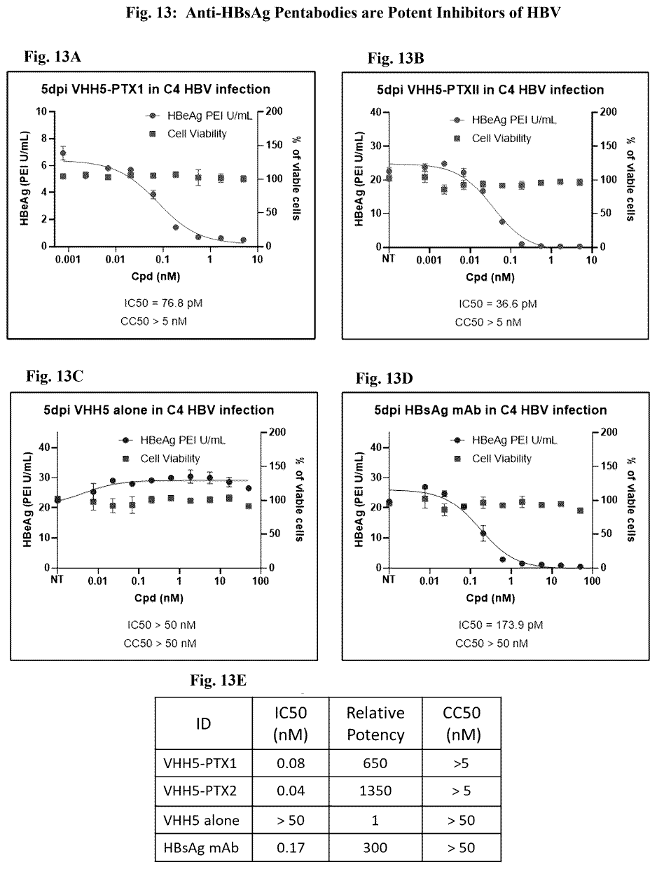

Results: These data demonstrate specific and potent Pentabody inhibition of HBV infection with no observed toxicity (CC50). FIGS. 13A and 13B show viral inhibition assays for anti-HBsAg Pentabodies on PTX-1 and PTX-2 chassis, respectively, with IC50's less than 100 μM obtained for each. The monomeric VHH5 (no PTX fusion) had an IC50 650-1350 fold higher than the Pentabodies, indicating significant potency increase by virtue of the pentameric presentation of VHH5 in the Pentraxin-based Pentabodies. This suggests significant avidity effects with Pentraxin-based Pentabodies. A control monoclonal antibody (IgG) against HBsAg had an IC50 of 174 μM, 2-4 fold less potent than the Pentabodies.

### Example 4

### Anti SARS-COV-2 Pentabodies Inhibit ACE-2 Binding

Method: This competitive ELISA measures the ability of anti SARS-COV-2 Pentabodies to compete with ACE-2 for binding to the receptor binding domain (RBD) of the SARS-COV-2 spike protein. In summary, a high-binding ELISA plate (Corning 3369) was coated with 100 μL of 2 μg/mL SARS-COV-2 RBD (Invitrogen RP-87678) overnight at 4° C. The next day, the plate was washed and blocked with 150 μL 1% BSA in PBS and incubated at 37° C. for 45 minutes. The plate was then washed and treated with 50 μL 2× Pentabody or Antibody and 50 μL of 0.5 ug/mL ACE2-biotin (SAE0171-25UG) at 37° C. for 1 hour. Inhibitor and sample dilutions were done in TBS. Wells were then washed and treated with 100 μL 1:2000 dilution of extravidin-peroxidase (Sigma Aldrich E2886-1ML) in PBS-T for 45 min at 37° C. Plates were then developed with 100 μL room temperature TMB-Ultra (Thermofisher 34028) for 5 minutes, then treated with 200 μL 0.2 M HCl to stop the reaction. Absorbance was measured at 450 nm and 630 nm. Percent inhibition=1-(background-corrected absorbance/average TBS absorbance for the plate). The “no spike” controlled for non-specific inhibitor binding to plate, while the TBS samples verified RBD-ACE2 interaction representing 100% ACE2-RBD binding. The wtPTX-2 sample demonstrated binding was mediated by VHH20, and the VHH5-PTX2 control confirms specificity of VHH20 to SARS-COV-2 Spike RBD.

Results: This experiment tested two linker designs (alpha=(A(EAAAK)₄A (SEQ ID NO: 10), flex=(GGGGS)₃ (SEQ ID NO: 11)) for anti-SARS-COV-2 VHH20-PTX2 Pentabodies against monomeric VHH20 and an anti-RBD Antibody. The data show that both anti-RBD pentabodies displaced ACE2 binding to RBD coated plates with IC50s <1 nM, while monomoric VHH20 bound with an IC50 of 3 nM and the anti RBD IgG with an IC50 of 50 nM. These results suggest the higher potency of pentameric presentation of binding activities over monomeric or IgG forms against the same targets.

### Example 5

### Dengue Virus Diagnostics

Method: This experiment demonstrates the ability to create a sensitive and specific diagnostic by labeling a Pentabody with an Alexafuor dye using NHS chemistry to covalently attach dye to surface lysines. In this example, anti-DENV-NS1 VHH sequences were obtained from literature (Fatima, A., 2014, PLOS One; Shriver-Lake L. C., 2018, Nature) to create DB5- and P2-PTX-2 pentabodies. The purified Pentabodies were labeled with NHS-Alexafluor 488 according to manufacturer's procedures, purified by phosphoethanolamine affinity chromatography to remove unreacted dye, and then used in a flow-cytometry format to detect NS1-linked beads.

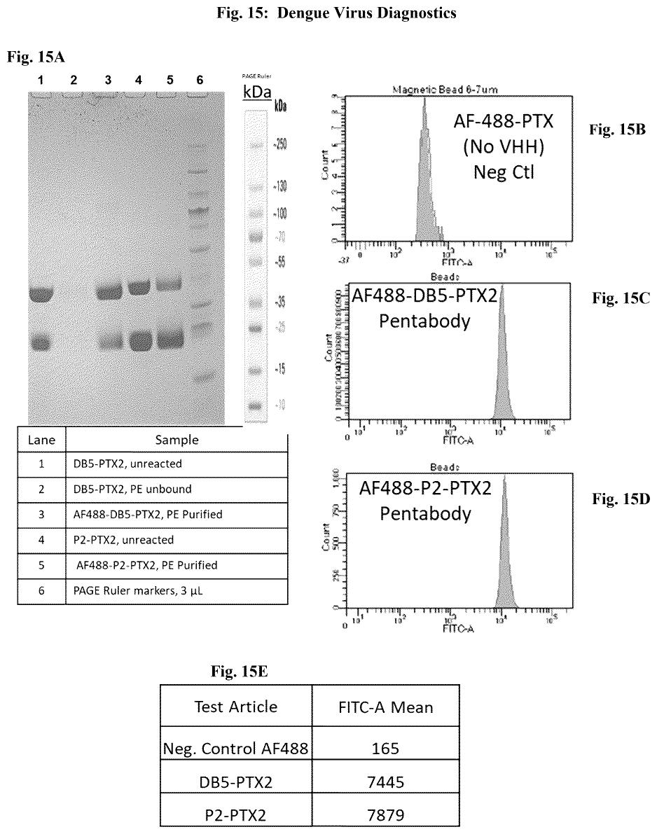

Results: FIG. 15A shows the purification and AF488-labeling of DB5- and P2-Pentabodies by SDS PAGE. UV-vis spectroscopy indicates approximately 5-fold molar excess of AF488: Pentabody (data not shown). FIGS. 15B, C, and D are flow cytometry data of the negative control (15B, native PTX-2 labeled with AF488, no VHH fusion), AF488-DB5-PTX2 (15C), and AF488-P2-PTX2 (15D). These plots show significant signal with mean FITC signals at least 15× above background as listed in the Table FIG. 15E. This confirms specific detection of DENV NS1 protein on flow cytometry beads, suggesting this is a viable path to a DENV diagnostic tool.

### Example 6

### Transglutaminase-Catalyzed Alexafluor Conjugation

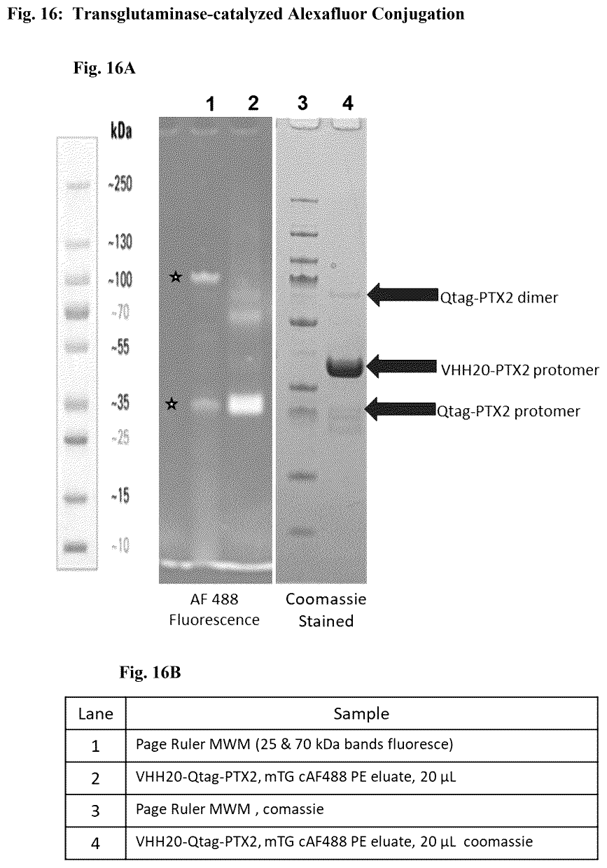

This example demonstrates the ability to specifically label PTX-based molecules using microbial transglutaminase (mTG) to covalently couple a primary-amine derivative (cadaverine) of AF-488 to a heteropentameric PTX2 molecule comprising N-terminally tagged protomers with a Q-tag sequence, an acceptor substrate for mTG, and anti-SARS COV-2 (VHH20) protomers. FIG. 16A shows the fluorescence of the Qtag protomers in lane two, while Lane 4 shows a coomassie-stained gel of the dye-labeled heteropentamer.

Briefly, 1.4 μM VHH20-Qtag-PTX2 heteropentamer and 70 μM cadaverine alexafluor 488 were subjected to catalysis with 70 nM mTG for 15 hours at room temperature in PBS, pH 7.0 buffer. Reaction products were purified from unreacted dye by purification over PE affinity resin and separated by SDS-PAGE. Fluorescent species in the gel were visualized using an iBright FL1500 gel imaging system (Thermofisher) and the gel was subsequently stained with Coomassie brilliant blue R250. The figure shows Qtag-PTX2 protomers were specifically conjugated to cAF488 while VHH20-PTX2 protomers were not. Heteropentameric Pentraxins such as this can be used as diagnostic reagents to detect target antigens

### Example 7

### Non-Covalent Calcium-Mediated Phophoethanolamine-Pentraxin Complexes

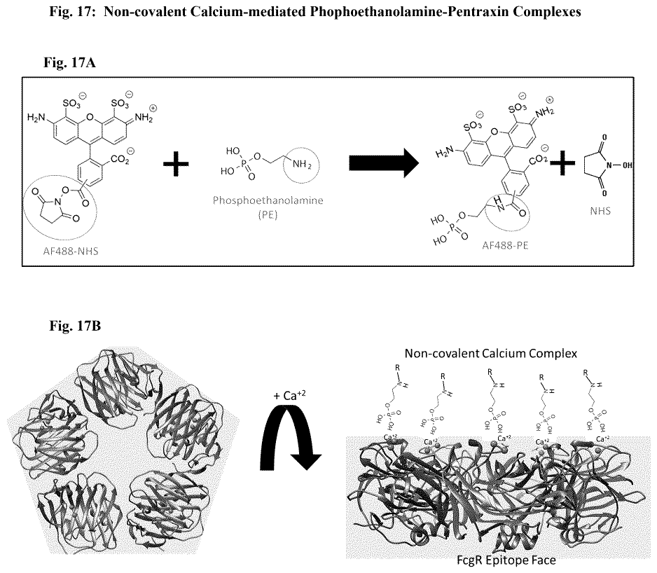

This example highlights the versatilility of engineering pentraxin molecules for specific purposes. In this case, the natural affinity of Pentraxins is exploited to generate non-covalent calcium complexes to any native pentraxin or engineered pentraxin fusion protein. Phosphoethanolamine (PE) is the cognate ligand for pentraxin-2 which also binds to pentraxin-1 with high affinity. Phosphoethanolamine is used as an affinity ligand by covalently coupling the amino group at one end of PE to agarose beads for use in column chromatography. In similar fashion, the amino group of PE can be coupled with any number of small molecules or peptides via NHS coupling chemistry. In this way, small molecules (payloads) can be non-covalently linked to the pentraxin chassis via calcium for delivery to a target via a pentabody. FIG. 17A shows a reaction scheme for covalently coupling NHS-AF488 to PE FIG. 17B displays a schematic model of amino-derivatized PE coupled via calcium to a pentraxin chassis.

### Example 8

### Immuno-Oncology/Autoimmune Applications of PTX Fusions

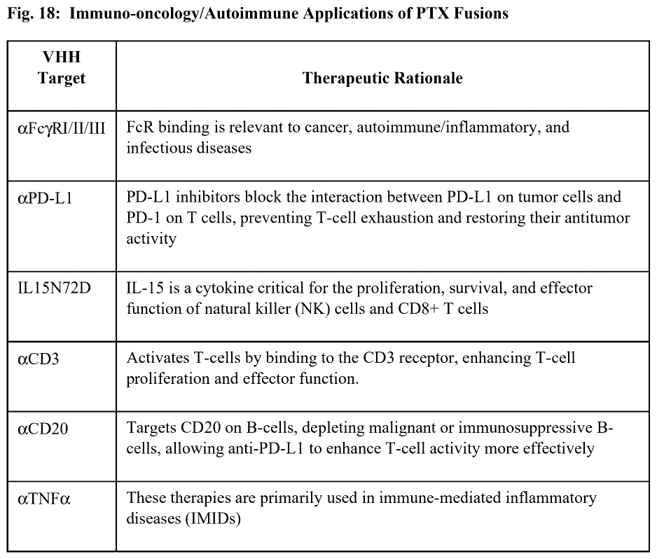

To adapt the Pentraxin-fusion technology to immuno-oncology or autoimmune applications, FcγR or T-cell surface receptors can be targeted by VHH domains. Additionally, cytokines, checkpoint inhibitors, to T-cell stimulators like IL-15N72D, can also be targeted via pentabody/pentakine heteropentamers. Heteropentameric designs can also be used as T-cell engagers by targeting specific cell surface proteins (e.g. HER2) with VHH domains along with T-cell stimulators like aCD3 or IL15N72D. These combinations may be synergistic, for example, combining aCD3 and aPD-L1 binding activities on heteropentabodies, or αPDL1 and IL15N72D may lead to very potent T-cell stimulators for oncology treatments. See FIG. 18.

### Example 9

### H5N1 Vaccine Development Using Pentraxin Proteins

The purpose of this experiment was to generate vaccines against Influenza A virus strain H5N1 by creating N-terminal fusions of selected H5N1 hemagglutinin (HA) and neuraminidase (NA) peptides to hPTX-2. The sequences of both H5N1 hemagglutinin and neuraminidase were examined for potential immunogenic epitopes (Dashti, F., 2024 Virology J. 21 (67), strain (A/duck/Shandong/093/2004 (H5N1) and several peptides were chosen as N-terminal fusion partners:

HA peptides:
1.
(SEQ ID NO: 12)
MPFHNIHPLTI (aa305-315)
2.
(SEQ ID NO: 13)
RTLDFHDSN (aa452-460)
3.
(SEQ ID NO: 14)
GWQGMVDGWYGYHHSNEQGSGYAADKESTQKAIDGVTNK (aa359-597)
NA peptides:
4.
(SEQ ID NO: 15)
NSSLCPI (aa68-74)
5.
(SEQ ID NO: 16)
AYGVKGFSFKY (aa347-357)

These PTX-2 fusion proteins were expressed in CHO cells by transient transfection and are tested for their ability to raise specific immune responses to Influenza virus H5N1. Additionally, these PTX-2 based pentagens are being formulated in the presence of calcium to induce colloidal suspensions, and these formulations are being compared to those lacking calcium to assess whether the colloidal suspension formulations (self-adjuvating) result in increased immunogenicity relative to formulations without by virtue of their particulate structures of repeating patterns.

### Example 10

### Pentabodies as T-Cell Engagers to HER2-Positive Breast Cancer

The purpose of this experiment was to create bispecific Pentabodies on the PTX-1 chassis to bind simultaneously to T cells (T-cell engagers) via an anti-CD3 VHH domain and to HER2-positive breast cancer cells via an anti-HER2 VHH domain on the same pentameric scaffold. V_{H}H domains, also known as nanobodies or single-domain antibodies are small (~12 kDa) single amino acid chain binding domains found naturally in camelid species (Llamas, Alpacas, Camels). Fused to the N-terminus of Pentraxin proteins, e.g. PTX-1 (CRP) or PTX-2 (SAP), these can be assembled in heteropentameric structures by virtue of the native pentameric structure of the pentraxin family of proteins. Heteropentameric pentabodies represent a potential improvement in the simplicity of design, ease of manufacture, and increase in potency over conventional IgG-based antibodies and bispecific mAbs due to the pentameric presentation of binding domains, while retaining the ability of the Pentraxin chassis (either PTX-1 or PTX2) to interact with immune cells via Fc receptor epitopes. This creates a novel class of antibody therapeutics able to bridge the innate and adaptive immune systems in patients.

### Example 11

Pentraxin Proteins with Dengue Virus Immunoreactivity

The purpose of this experiment was to design and generate pentraxin-based vaccines, or Pentagens, against Dengue virus by N-terminal fusion of peptide sequences from the NS1 surface protein found on Dengue viruses. The sequence of the NS1 protein was analyzed by Fatima, et al (Fatima, A., 2014, PLOS one 9 (4)), and several immunogenic determinants were identified from the DENV type 1 NS1 sequence:

6.
(SEQ ID NO: 17)
DSGCVINWKGRELKCG (aa 1-16)
7.
(SEQ ID NO: 18)
VTNEVHTWTEQYKFQ (aa 21-35)
8.
(SEQ ID NO: 19)
HKYSWKSWGKAKIIG (aa 111-125)

These peptide sequences were fused to the N-terminus of PTX-2 and expressed by transient transfection of CHO cells and purified to >90% purity (SDS-PAGE) by phosphoethanolamine affinity chromatography. These proteins will ultimately be assessed for their ability to generate a specific immune response to Dengue virus type 1 in an appropriate animal model.

Another approach may be to fuse longer sequences that may enable presentation of multiple antigenic epitopes, or more extensive tertiary structure elements to be presented as antigens to the immune system. For example, the following two sequences may confer more robust immune responses against DENV NS1 (dNS1, Nasar, S., 2024, Nature) or Envelope proteins (EDIII, Chen, W-H, 2017, Virol. J), respectively:

9. dNS1 (SEQ ID NO: 20):
EVEDYGFGIFTTNIWLKLRDSYTQVCDHRLMSAAIKDSKAVHADMGYWIESEKN
ETWKLARASFIEVKTCIWPKSHTLWSNGVLESEMIIPKIYGGPISQHNYRPGYFTQ
TAGPWHLGKLELDFDLCEGTTVVVDEHCGNRGPSLRTTTVTGKIIHEWCC
10. EDIII (SEQ ID NO: 21):
GMSYVMCTGSFKLEKEVAETQHGTVLVQVKYEGTDAPCKIPFSTQDEKGVTQN
GRLITANPIVTDKEKPVNIEAEPPFGESYIVVGAGEKALKLSWFKKG

We claim:

1. A composition comprising a recombinant human Pentraxin-1/2 (rhPTX-1 or rhPTX-2; rhPTX-1/2) pentamer having from one to five protomers linked to a fusion partner.

2. The composition of claim 1, wherein the rhPTX-1/2 pentamer comprises from one to four unmodified, native rhPTX-1/2 protomers.

3. The composition of claim 1, wherein the fusion partner is fused to the N- or C-terminus of rhPTX-1/2 either with or without an intervening peptide linker.

4. The composition of claim 1, wherein the fusion partner is a camelid V_{H}H domain engineered to bind a target antigen.

5. The composition of claim 1, wherein the fusion partner is an antigen derived from an infectious pathogen.

6. The composition of claim 1, wherein the fusion partner is a cytokine.

7. The composition of claim 1, wherein the fusion partner is an enzyme.

8. The composition of claim 1, wherein the fusion partner is an annexin or an annexin fragment.

9. The composition of claim 1, wherein the fusion partner is either an agonist, antagonist, inhibitor, cell internalization, or cell-signaling peptide, for example, Fas ligand.

10. The composition of claim 1, wherein the rhPTX-1/2 pentamer comprises from one to five protomers with any combination of distinct fusion partners.

11. The composition of claim 1, wherein the rhPTX-2 pentamer comprises from one to five protomers that are modified to remove the consensus glycosylation sequence resulting in N-linked glycosylation at position 32.

12. The composition of claim 1, wherein the Fc receptor binding epitopes of the rhPTX-1/2 chassis have been engineered to modulate (either increasing or decreasing) the binding affinity to selected Fcα, Fcγ, or FcRn receptors in order to obtain a desired signaling outcome, e.g., but not limited to, enhanced dendritic cell (DC), T cell, or NK cell activation, or extended circulating half-life.

13. The composition of claim 1, wherein the fusion partner is a known therapeutic agent, or the target of a therapeutic agent.

14. The composition of claim 1, wherein a small molecule payload is covalently coupled to one or more pentraxin protomers either by chemical or enzymatic means, resulting in a pentraxin-based antibody drug conjugate therapeutic, or a diagnostic agent when the small molecule payload is a fluorescent dye or reporter group.

15. The composition of claim 1, further comprising one or more pharmaceutically acceptable active ingredients, carriers, or excipients.

16. The composition of claim 1, wherein the composition comprises a vaccine.

17. The composition of claim 16, wherein the recombinant human Pentraxin-1/2 consists of rhPTX-2.

18. A method for preparing rhPTX-1/2 pentameric fusion proteins comprising: introducing a DNA or RNA coding sequence into an expression system host cell such that the cell is able to produce the rhPTX-1/2 fusion protein of interest, isolating and purifying the expressed rhPTX-1/2 pentameric fusion proteins, and formulating the purified protein in a pharmaceutically acceptable form for administration to a patient or subject.

19. The method of claim 18 further comprising: introduction into an expression system host cell of from one to five distinct DNA or RNA coding regions for rhPTX-1/2 protomers resulting in expression of rhPTX-1/2 pentameric fusion proteins containing from one to five distinct protomers, including unmodified, native rhPTX-1/2 protomers.

20. A method for expressing rhPTX-1/2 fusion proteins in situ in a patient or subject using any suitable gene therapy or cellular therapy technology such that the desired therapeutic fusion protein is produced by the patient or subject wherein the therapeutic effect is manifested.


SEQUENCE LISTING (21 sequences)

SEQ ID NO: 1 - protein, 227 aa, Homo sapiens
QTDMEKLLCFLVLTSLSHAFGQTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 2 - protein, 204 aa, Homo sapiens
HTDLSGKVFVFPRESVTDHVNLITPLEKPLQNFTLCFRAYSDLSRAYSLFSYNTQGRDNELLVYKERVGEYSLYIGRHKVTSKVIEKFPAPVHICVSWESSSGIAEFWINGTPLVKKGLRQGYFVEAQPKIVLGQEQDSYGGKFDRSQSFVGEIGDLYMWDSVLPPENILSAYQGTPLPANILDWQALNYEIRGYVIIKPLVWV

SEQ ID NO: 3 - protein, 204 aa, synthetic construct; identical to SEQ ID NO: 2

SEQ ID NO: 4 - protein, 204 aa, synthetic construct; identical to SEQ ID NO: 2

SEQ ID NO: 5 - protein, 5 aa, synthetic construct
  REPEAT 1..5: note = 2-6 repeats
GGGGS

SEQ ID NO: 6 - protein, 7 aa, synthetic construct
  REPEAT 2..6: note = 2-6 repeats
AEAAAKA

SEQ ID NO: 7 - protein, 5 aa, synthetic construct
KLAAA

SEQ ID NO: 8 - protein, 18 aa, synthetic construct
KESGSVSSEQLAQFRSLD

SEQ ID NO: 9 - protein, 14 aa, synthetic construct
EGKSSGSGSESKST

SEQ ID NO: 10 - protein, 7 aa, synthetic construct
  REPEAT 2..6: note = 4 repeats
AEAAAKA

SEQ ID NO: 11 - protein, 5 aa, synthetic construct
  REPEAT 1..5: note = 3 repeats
GGGGS

SEQ ID NO: 12 - protein, 11 aa, Alphainfluenzavirus influenzae
MPFHNIHPLTI

SEQ ID NO: 13 - protein, 9 aa, Alphainfluenzavirus influenzae
RTLDFHDSN

SEQ ID NO: 14 - protein, 39 aa, Alphainfluenzavirus influenzae
GWQGMVDGWYGYHHSNEQGSGYAADKESTQKAIDGVTNK

SEQ ID NO: 15 - protein, 7 aa, Alphainfluenzavirus influenzae
NSSLCPI

SEQ ID NO: 16 - protein, 11 aa, Alphainfluenzavirus influenzae
AYGVKGFSFKY

SEQ ID NO: 17 - protein, 16 aa, Dengue virus
DSGCVINWKGRELKCG

SEQ ID NO: 18 - protein, 15 aa, Dengue virus
VTNEVHTWTEQYKFQ

SEQ ID NO: 19 - protein, 15 aa, Dengue virus
HKYSWKSWGKAKIIG

SEQ ID NO: 20 - protein, 160 aa, Dengue virus
EVEDYGFGIFTTNIWLKLRDSYTQVCDHRLMSAAIKDSKAVHADMGYWIESEKNETWKLARASFIEVKTCIWPKSHTLWSNGVLESEMIIPKIYGGPISQHNYRPGYFTQTAGPWHLGKLELDFDLCEGTTVVVDEHCGNRGPSLRTTTVTGKIIHEWCC

SEQ ID NO: 21 - protein, 100 aa, Dengue virus
GMSYVMCTGSFKLEKEVAETQHGTVLVQVKYEGTDAPCKIPFSTQDEKGVTQNGRLITANPIVTDKEKPVNIEAEPPFGESYIVVGAGEKALKLSWFKKG